SEQUENCE LISTING (37 sequences)

SEQ ID NO: 1 - protein, 184 aa, Homo sapiens
  REGION 1..184: note = Gremlin-1
MSRTAYTVGALLLLLGTLLPAAEGKKKGSQGAIPPPDKAQHNDSEQTQSPQQPGSRNRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 2 - protein, 168 aa, Homo sapiens
  REGION 1..168: note = Gremlin-2
MFWKLSLSLFLVAVLVKVAEARKNRPAGAIPSPYKDGSSNNSERWQHQIKEVLASSQEALVVTERKYLKSDWCKTQPLRQTVSEEGCRSRTILNRFCYGQCNSFYIPRHVKKEEESFQSCAFCKPQRVTSVLVELECPGLDPPFRLKKIQKVKQCRCMSVNLSDSDKQ

SEQ ID NO: 3 - protein, 8 aa, synthetic construct
  REGION 1..8: note = CDR1 HC Var 3, 5, 7, 11, 13
GYSFTGYY

SEQ ID NO: 4 - protein, 8 aa, synthetic construct
  REGION 1..8: note = CDR2 HC Var 5, 7, 13
FFPYSGFS

SEQ ID NO: 5 - protein, 12 aa, synthetic construct
  REGION 1..12: note = CDR3 HC Var 3, 5, 7, 11, 13
ARGGLGRGYFDV

SEQ ID NO: 6 - protein, 6 aa, synthetic construct
  REGION 1..6: note = CDR1 LC Var 3, 5, 7
DHINNW

SEQ ID NO: 7 - skipped

SEQ ID NO: 8 - protein, 9 aa, synthetic construct
  REGION 1..9: note = CDR3 LC Var 3, 5, 7, 11, 13
QQYWSSPRT

SEQ ID NO: 9 - protein, 8 aa, synthetic construct
  REGION 1..8: note = CDR2 HC Var 3, 11
FFPYNGFS

SEQ ID NO: 10 - protein, 6 aa, synthetic construct
  REGION 1..6: note = CDR1 LC Var 11, 13
DHISNW

SEQ ID NO: 11 - protein, 123 aa, synthetic construct
  REGION 1..123: note = HC Var 3
EVQLVQSGPEVVKPGASVKVSCKASGYSFTGYYMHWVRQAPGQGLEWMGYFFPYNGFSNYAQKFQGRVTLTVDKSKSTAYMELSRLRSEDTATYYCARGGLGRGYFDVWGQGTLVTVSSASTK

SEQ ID NO: 12 - protein, 123 aa, synthetic construct
  REGION 1..123: note = HC Var 5
EVQLVQSGPEVVKPGASVKVSCKASGYSFTGYYMHWVKQAPGQGLEWMGYFFPYSGFSNYAQKFQGRVTLTVDKSISTAYMELSRLRSEDTAVYYCARGGLGRGYFDVWGQGTLVTVSSASTK

SEQ ID NO: 13 - protein, 123 aa, synthetic construct
  REGION 1..123: note = HC Var 7
EVQLVQSGPEVVKPGASVKVSCKASGYSFTGYYMHWVRQAPGQGLEWMGYFFPYSGFSNYAQKFQGRVTLTVDKSKSTAYMELSRLRSEDTATYYCARGGLGRGYFDVWGQGTLVTVSSASTK

SEQ ID NO: 14 - protein, 123 aa, synthetic construct; identical to SEQ ID NO: 11
  REGION 1..123: note = HC Var 11

SEQ ID NO: 15 - protein, 123 aa, synthetic construct; identical to SEQ ID NO: 12
  REGION 1..123: note = HC Var 13

SEQ ID NO: 16 - protein, 107 aa, synthetic construct
  REGION 1..107: note = LC Var 3
DIQMTQSPSSLSASLGDRVTITCKASDHINNWLAWYQQKPGKAPRLLISGATSLETGVPSRFSGSGSGTDYTLTISSLQPEDVATYYCQQYWSSPRTFGGGTKLEIK

SEQ ID NO: 17 - protein, 107 aa, synthetic construct; identical to SEQ ID NO: 16
  REGION 1..107: note = LC Var 5

SEQ ID NO: 18 - protein, 107 aa, synthetic construct; identical to SEQ ID NO: 16
  REGION 1..107: note = LC Var 7

SEQ ID NO: 19 - protein, 107 aa, synthetic construct
  REGION 1..107: note = LC Var 11
DIQMTQSPSSLSASLGDRVTITCKASDHISNWLAWYQQKPGKAPRLLISGATSLETGVPSRFSGSGSGTDYTLTISSLQPEDVATYYCQQYWSSPRTFGGGTKLEIK

SEQ ID NO: 20 - protein, 107 aa, synthetic construct; identical to SEQ ID NO: 19
  REGION 1..107: note = LC Var 13

SEQ ID NO: 21 - DNA, 369 bp, synthetic construct
  misc_feature 1..369: note = HC Var 3
  source 1..369: mol_type = other DNA
gaagttcagctggtgcagtctggccccgaggttgtgaaacctggcgcttctgtgaaggtgtcctgcaaggcctccggctactctttcaccggctactacatgcactgggtccgacaggctccaggacagggacttgagtggatgggctacttcttcccctacaacggcttctccaactacgcccagaaattccagggcagagtgaccctgaccgtggacaagtctaagtccaccgcctacatggaactgtcccggctgagatctgaggacaccgccacctactactgtgctagaggcggactcggcagaggctactttgatgtgtggggccagggcaccctggtcacagtttctagcgcttccacaaag

SEQ ID NO: 22 - DNA, 369 bp, synthetic construct
  misc_feature 1..369: note = HC Var 5
  source 1..369: mol_type = other DNA
gaagttcagctggtgcagtctggccccgaggttgtgaaacctggcgcttctgtgaaggtgtcctgcaaggcctccggctactctttcaccggctactacatgcactgggtcaagcaggcccctggacagggacttgagtggatgggctacttcttcccctactccggcttctccaactacgcccagaaattccagggcagagtgaccctgaccgtggacaagtctatctccaccgcctacatggaactgtcccggctgagatctgaggacaccgccgtgtactactgtgctagaggcggactcggcagaggctactttgatgtgtggggccagggcaccctggtcacagtttctagcgcttccacaaag

SEQ ID NO: 23 - DNA, 369 bp, synthetic construct
  misc_feature 1..369: note = HC Var 7
  source 1..369: mol_type = other DNA
gaagttcagctggtgcagtctggccccgaggttgtgaaacctggcgcttctgtgaaggtgtcctgcaaggcctccggctactctttcaccggctactacatgcactgggtccgacaggctccaggacagggacttgagtggatgggctacttcttcccctactccggcttctccaactacgcccagaaattccagggcagagtgaccctgaccgtggacaagtctaagtccaccgcctacatggaactgtcccggctgagatctgaggacaccgccacctactactgtgctagaggcggactcggcagaggctactttgatgtgtggggccagggcaccctggtcacagtttctagcgcttccacaaag

SEQ ID NO: 24 - DNA, 369 bp, synthetic construct; identical to SEQ ID NO: 21
  misc_feature 1..369: note = HC Var 11
  source 1..369: mol_type = other DNA

SEQ ID NO: 25 - DNA, 369 bp, synthetic construct; identical to SEQ ID NO: 22
  misc_feature 1..369: note = HC Var 13
  source 1..369: mol_type = other DNA

SEQ ID NO: 26 - DNA, 321 bp, synthetic construct
  misc_feature 1..321: note = LC Var 3
  source 1..321: mol_type = other DNA
gacatccagatgacccagtctccatcctctctgtctgcttccctgggcgacagagtgaccatcacatgcaaggcctccgaccacatcaacaactggctggcctggtatcagcagaagcctggcaaggctccccggctgttgatttctggcgctacctctctggaaaccggcgtgccctctagattctccggctctggctctggcaccgactataccctgacaatctccagcctgcagcctgaggatgtggccacctactactgccagcagtactggtcctctcctcggacatttggcggaggcaccaagctggaaatcaag

SEQ ID NO: 27 - DNA, 321 bp, synthetic construct; identical to SEQ ID NO: 26
  misc_feature 1..321: note = LC Var 5
  source 1..321: mol_type = other DNA

SEQ ID NO: 28 - DNA, 321 bp, synthetic construct; identical to SEQ ID NO: 26
  misc_feature 1..321: note = LC Var 7
  source 1..321: mol_type = other DNA

SEQ ID NO: 29 - DNA, 321 bp, synthetic construct
  misc_feature 1..321: note = LC Var 11
  source 1..321: mol_type = other DNA
gacatccagatgacccagtctccatcctctctgtctgcttccctgggcgacagagtgaccatcacatgcaaggcctccgaccacatctccaactggctggcctggtatcagcagaagcctggcaaggctccccggctgttgatttctggcgctacctctctggaaaccggcgtgccctctagattctccggctctggctctggcaccgactataccctgacaatctccagcctgcagcctgaggatgtggccacctactactgccagcagtactggtcctctcctcggacatttggcggaggcaccaagctggaaatcaag

SEQ ID NO: 30 - DNA, 321 bp, synthetic construct; identical to SEQ ID NO: 29
  misc_feature 1..321: note = LC Var 13
  source 1..321: mol_type = other DNA

SEQ ID NO: 31 - protein, 120 aa, synthetic construct
  REGION 1..120: note = 14-D10-2 HC
EVQLQQSGPELVKPGASVKISCKASGYSFTGYYMHWVKQSHGNILDWIGYFFPYNGFSNCNQKFKGKATLTVDKSSSTAYMELRSLTSEDSAVYYCARGGLGRGYFDVWGTGTTVTVSSA

SEQ ID NO: 32 - DNA, 360 bp, synthetic construct
  misc_feature 1..360: note = 14-D10-2 HC
  source 1..360: mol_type = other DNA
gaagtgcagctgcagcagtctggacctgagctggtgaagcctggggcttcagtgaagatatcctgcaaggcttctggttactcattcactggctactacatgcactgggtgaaacagagccatggaaatatcctcgattggattggatatttttttccttacaatggtttttctaactgcaaccagaaattcaagggcaaggccacattgactgtagacaagtcctctagcacagcctacatggagctccgcagcctgacatctgaggactctgcagtctattactgtgcaagagggggactgggacggggatacttcgatgtctggggcacagggaccacggtcaccgtctcctcagcc

SEQ ID NO: 33 - protein, 107 aa, synthetic construct
  REGION 1..107: note = 14-D10-2 LC
DIQMTQSPSYLSVSLGGRVTITCKASDHINNWLAWYQQKPGNAPRLLISGATSLETGVPSRFSGSGSGKDYTLSITSLQTEDVATYYCQQYWSSPRTFGGGTKLEIK

SEQ ID NO: 34 - DNA, 321 bp, synthetic construct
  misc_feature 1..321: note = 14-D10-2 LC
  source 1..321: mol_type = other DNA
gatattcagatgacccagtctccatcctacttgtctgtatctctaggaggcagagtcaccattacttgcaaggcaagtgaccacattaataattggttagcctggtatcagcagaaaccaggaaatgctcctaggctcttaatatctggtgcaaccagtttggaaactggggttccttcaagattcagtggcagtggatctggaaaggattacactctcagcattaccagtcttcagactgaagatgttgctacttattactgtcaacagtattggagtagtcctcggacgttcggtggaggcaccaagctggaaatcaag

SEQ ID NO: 35 - protein, 17 aa, Homo sapiens
  REGION 1..17: note = huGREM1_105-121
EEGCNSRTIINRFCYGQ

SEQ ID NO: 36 - protein, 17 aa, Homo sapiens
  REGION 1..17: note = huGREM2_84-100
EEGCRSRTILNRFCYGQ

SEQ ID NO: 37 - protein, 18 aa, synthetic construct
  REGION 1..18: note = MYH6_614 629
RSLKLMATLFSTYASADR

(19) United States
(12) Patent Application Publication
(10) Pub. No.: US 2025/0257123 A1
(43) Pub. Date: Aug. 14, 2025

(54) ANTIBODIES OR ANTIGEN-BINDING FRAGMENTS PAN-SPECIFICALLY BINDING TO GREMLIN-1 AND GREMLIN-2 AND USES THEREOF

(71) Applicant: KANTONSSPITAL ST. GALLEN, St. Gallen (CH)

(72) Inventors: Hung-Wei CHENG, St. Gallen (CH); Cristina del Carmen GIL CRUZ, St. Gallen (CH); Christian Ivan PÉREZ SHIBAYAMA, St. Gallen (CH); Lucas ONDER, Dornbirn (AT); Burkhard LUDEWIG, St. Gallen (CH)

(21) Appl. No.: 18/857,007

(22) PCT Filed: Apr. 20, 2023
(86) PCT No.: PCT/EP2023/060378

§ 371 Date: Oct. 15, 2024

(30) Foreign Application Priority Data
Apr. 20, 2022 (EP) 22169123.1

(51) Int. Cl. C07K 16/18; A61K 39/00; A61K 45/06; A61P 9/00; A61P 29/00

(52) U.S. Cl. CPC C07K 16/18; A61K 45/06; A61P 9/00; A61P 29/00; A61K 2039/505; C07K 2317/24; C07K 2317/565; C07K 2317/76; C07K 2317/94

(57) ABSTRACT

The invention relates to antibodies, or antigen-binding fragments thereof, pan-specifically binding to Gremlin-1 and Gremlin-2. The antibodies described herein may be humanized antibodies and/or deimmunized antibodies. Means and methods provided herein may be used in treating and/or preventing heart failure and/or an inflammatory disease, in particular an inflammatory disease of the heart. Also encompassed by the invention are polynucleotides encoding the antibodies, or antigen-binding fragments thereof, host cells comprising the polynucleotides of the invention, methods for producing the antibodies, or antigen-binding fragments thereof, and pharmaceutical compositions comprising the antibodies, or antigen-binding fragments thereof.

### RELATED APPLICATIONS

This application is a 35 U.S.C. § 371 filing of International Patent Application No. PCT/EP2023/060378, filed Apr. 20, 2023, which claims priority to European Patent Application No. 22169123.1, filed Apr. 20, 2022, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Oct. 15, 2024, is named 756617_VOS9-022US_ST26.xml and is 51,555 bytes in size.

The invention relates to antibodies, or antigen-binding fragments thereof, pan-specifically binding to Gremlin-1 and Gremlin-2. The antibodies described herein may be humanized antibodies and/or deimmunized antibodies. Means and methods provided herein may be used in treating and/or preventing heart failure and/or an inflammatory disease, in particular an inflammatory disease of the heart. Also encompassed by the invention are polynucleotides encoding the antibodies, or antigen-binding fragments thereof, host cells comprising the polynucleotides of the invention, methods for producing the antibodies, or antigen-binding fragments thereof, and pharmaceutical compositions comprising the antibodies, or antigen-binding fragments thereof.

Gremlin-1 is an inhibitor in the bone morphogenic protein (BMP) signalling pathway. Gremlin-1 primarily inhibits bone morphogenesis and is described to be implicated in disorders of increased bone formation and several cancers. Antibodies that bind to Gremlin-1 are known in the art. Such antibodies have been described as being suitable for use in the treatment of certain diseases such as cancer (WO 2019/243801) or bone fractures or defects (WO 2019/158658).

Inflammatory diseases and heart failure are a major health and economic burden for society.

Heart failure is a cardiac condition that occurs when a problem with the structure and/or function of the heart impairs its ability to supply sufficient blood flow to meet the body's needs.

Myocarditis is an inflammatory heart disease and one of the most common causes of myocardial damage in patients with suspected myocardial infarction but non-obstructive coronary arteries (Bier, E., and De Robertis, E. M., 2015, Science 348, aaa5838). Patients suffering from acute myocarditis may present to the clinic with acute heart failure, infarct-like symptoms and/or arrhythmic events (Brazil, D. P. et al., 2015, Trends Cell Biol 25, 249-264). Acute cardiac inflammation develops into lethal inflammatory cardiomyopathy in 20-30% of the patients (Caforio, A. L. et al., 2013, Eur Heart J 34, 2636-2648, 2648a-2648d; Csiszar, A. et al. 2005, Circulation 111, 2364-2372).

The formation of inflammatory foci in the heart tissue not only contributes to the eradication of pathogens in the case of infection, but also impacts cardiac remodelling through the promotion of tissue reparation by activating fibroblastic stromal cells, which constitute approximately 20% of non-cardiomyocytic cells in the healthy heart (Pinto, A. R et al., 2016. Circ Res 118, 400-409). Fibroblast activation after injury plays a pivotal part in the repair process because these cells coordinate revascularization and remodelling of the tissue leading to restoration and preservation of the cardiac architecture and function. However, fibroblast function needs to be tightly controlled because exacerbated fibroblast activity, e.g. during chronic inflammation, results in fibrosis. Fibrotic damage in the heart is characterized by an excess of extracellular matrix deposition, which leads to increased stiffness and reduced compliance of the tissue, cardiomyocyte damage and ultimately heart failure (Diez, J. et al., 2020, J Am Coll Cardiol 75, 2204-2218).

The interaction of immune cells with any given tissue, e.g. the heart tissue, is regulated by a plethora of surface molecules and soluble factors that act at short range. Tissue cytokines belonging to the transforming growth factor-β (TGF-β) superfamily are of particular importance for the homeostasis and functional preservation of the cardiac tissue (Hanna, A., and Frangogiannis, N. G., 2019, Front Cardiovasc Med 6, 140.).

Bone morphogenetic proteins (BMPs), which are named according to their potential to induce bone and cartilage development (Wozney, J. M. et al., 1988, Science 242, 1528-1534), are the largest group in the TGF-β superfamily.

BMPs play essential roles as morphogens during early embryonic development, e.g. through patterning of the dorsoventral body axis (Bier, E., and De Robertis, E. M., 2015, Science 348, aaa5838). Mature BMPs are secreted to the extracellular space and form extracellular matrix-associated dimers before they bind to BMP receptor types 1 and 2 in an active hetero-tetrameric signalling complex (Brazil, D. P. et al., 2015, Trends Cell Biol 25, 249-264). Biological responses induced by BMP signalling are regulated through specific antagonists, i.e. the DAN-family proteins Gremlin-1 and Gremlin-2. Gremlin-1 is a 184 amino acid glycoprotein that binds with high affinity to BMP2, BMP4 and BMP7 and regulates BMP activity during development, controlling limb and kidney formation (Yanagita M. Cytokine Growth Factor Rev 16: 309-317, Khokha M K et al., 2003 Nat Genet., 34(3):303-7., Michos O et al., 2004, Development, 131(14):3401-10). Both BMP2 and BMP4 are essential factors for cardiac development (Jiao, K. et al., 2003, Genes Dev 17, 2362-2367; Ma, L. et al., 2005, Development 132, 5601-5611). Moreover, these tissue cytokines are involved in the remodelling process of the injured heart (Rutkovskiy, A. et al., 2017, Journal of the American Heart Association vol. 6, 9 e006339; Wu, X. et al., 2014, Life Sci 97, 145-154). It has been shown that the soluble inhibitor molecule Gremlin-2, which binds both BMP2 and BMP4, provides a molecular barrier that controls the magnitude and extent of inflammatory cell infiltration by suppressing canonical BMP signalling (Sanders, L. N. et al., 2016, Circ Res 119, 434-449). Inflammatory factors such as TNF lead to an increased production of BMP2 (Csiszar, A. et al. 2005, Circulation 111, 2364-2372.), which in turn stimulates the generation of the inhibitor Gremlin-2 (Sanders, L. N. et al., 2016, Circ Res 119, 434-449).

Pericardial inflammation is relatively frequent in clinical practice. Pericarditis is responsible for 0.1% of all hospital admissions and 5% of emergency room admissions due to chest pain. Data collected from a Finnish national registry (2000-2009) showed an incidence rate of hospitalizations of 3.3 per 100 000 person-year. (Adler, Yehuda et al. European heart journal vol. 36, 42 (2015): 2921-2964.).

The pericardium consists in a double-layered, elastic sac that covers the heart. Pericarditis is the most common pathologic process involving the pericardium. Pericarditis is characterized by inflammation of the pericardial sac and frequently, is accompanied by the accumulation of fluid within the pericardial sac that can compress the cardiac chambers limiting diastolic filling (Little, William C, and Gregory L Freeman. Circulation vol. 113, 12 (2006): 1622-32., Spodick, David H. The New England journal of medicine vol. 349, 7 (2003): 684-90.). The etiology of pericarditis is diverse and is influenced by the geographical/social environments. In developing countries, pericarditis is often secondary to tuberculosis, while in developed countries, pericarditis is more often related to autoinflammatory processes that might be triggered by pericardial injury such as radiotherapy or cardiac surgery (Imazio, Massimo et al. Journal of cardiovascular medicine (Hagerstown, Md.) vol. 10, 3 (2009): 217-30.). Importantly in the recent years, several reports revealed an association of acute pericarditis and myocarditis in patients with COVID-19 or that had received COVID-19 vaccination (Patone, Martina et al. Nature medicine vol. 28, 2 (2022): 410-422.; Diaz-Arocutipa, Carlos et al. Journal of cardiovascular medicine (Hagerstown, Md.) vol. 22, 9 (2021): 693-700.)

The clinical manifestations of pericarditis are heterogeneous spanning from self-limiting acute disease with complete resolution, incessant pericarditis, whose symptoms continue without interruption even for months after the first episode, or to recurrent pericarditis (Adler, Yehuda et al. European heart journal vol. 36, 42 (2015): 2921-2964.; Dababneh, Ehab. and Momin S. Siddique. StatPearls, 2021.). Recurrent pericarditis is a disease characterized by chronic and debilitating pericardial inflammation, with wide ranging effects on physical function, wellbeing and productivity accompanied with prolonged need of health care resources. Importantly, approximately 15 to 30% of the patients who suffer from an initial pericarditis episode will have a recurrence despite treatment with colchicine (Imazio, Massimo et al. Journal of cardiovascular medicine (Hagerstown, Md.) vol. 8, 10 (2007): 830-4.). Moreover, the increase of relapses increases by up to 50% in patients who had received corticosteroid therapy as treatment during the primary episode. Importantly, these therapies are associated with side-effects due to non-specific immunosuppression particularly when used in long-term (Ammirati, Enrico et al. Frontiers in medicine vol. 9 838564. 2022; Klein, Allan L et al. The New England journal of medicine vol. 384, 1 (2021): 31-41).

However, despite all the advances in the development of therapeutic interventions in inflammatory diseases, treatment efficacy and efficiency for these diseases often are limited and include physical activity, antiphlogistic treatment for patients with pericardial involvement, general heart failure treatment to immunosuppressive intervention (Caforio, A. L. et al., 2013, Eur Heart J 34, 2636-2648, 2648a-2648d.; Maisch, B., and P. Alter, 2018, Herz 43.5: 423-430).

Thus, there is a need for improved means and methods for treating Gremlin-1 and Gremlin-2 related diseases or disorders.

The above technical problem is solved by the embodiments disclosed herein and as defined in the claims.

Accordingly, the invention relates to, inter alia, the following embodiments:
1. An antibody, or an antigen-binding fragment thereof, pan-specifically binding to Gremlin-1 and Gremlin-2.
2. The antibody, or antigen-binding fragment thereof, according to embodiment 1, wherein the antibody or antigen-binding fragment thereof, binds to Gremlin-1 within the amino acid sequence SEQ ID NO: 1 and/or to Gremlin-2 within the amino acid sequence SEQ ID NO: 2.
3. The antibody, or antigen-binding fragment thereof, according to embodiment 2, wherein the antibody or antigen-binding fragment thereof, binds to Gremlin-1 within the amino acid sequence SEQ ID NO: 1 and to Gremlin-2 within the amino acid sequence SEQ ID NO: 2.
4. The antibody, or antigen-binding fragment thereof, according to any one of the embodiments 1 to 3, comprising at least one CDR as defined by the SEQ ID NO: 4.
5. The antibody, or antigen-binding fragment thereof, according to any one of the embodiments 1 to 3, comprising at least one CDR as defined by the SEQ ID NO: 10.
6. The antibody, or antigen-binding fragment thereof, according to any one of embodiments 1 to 5, comprising
   (a) a variable heavy (VH) chain comprising CDR1 as defined in SEQ ID NO: 3, CDR2 as defined in SEQ ID NO: 4 and CDR3 as defined in SEQ ID NO: 5 and a variable light (VL) chain comprising CDR1 as defined in SEQ ID NO: 6, CDR2 as is GAT and CDR3 as defined in SEQ ID NO: 8;
   (b) a variable heavy (VH) chain comprising CDR1 as defined in SEQ ID NO: 3, CDR2 as defined in SEQ ID NO: 9 and CDR3 as defined in SEQ ID NO: 5 and a variable light (VL) chain comprising CDR1 as defined in SEQ ID NO: 10, CDR2 is GAT and CDR3 as defined in SEQ ID NO: 8; or
   (c) a variable heavy (VH) chain comprising CDR1 as defined in SEQ ID NO: 3, CDR2 as defined in SEQ ID NO: 4 and CDR3 as defined in SEQ ID NO: 5 and a variable light (VL) chain comprising CDR1 as defined in SEQ ID NO: 10, CDR2 is GAT and CDR3 as defined in SEQ ID NO: 8.
7. The antibody, or antigen-binding fragment thereof, according to any one of embodiments 1 to 6, wherein the antibody or the antigen-binding fragment thereof comprises
   (a) a variable heavy (VH) chain sequence comprising the amino acid sequence of SEQ ID NO: 12 or a sequence having at least 90%, preferably at least 95% sequence identity to SEQ ID NO: 12; and
   a variable light (VL) chain sequence comprising the amino acid sequence of SEQ ID NO: 17 or a sequence having at least 90%, preferably at least 95% sequence identity to SEQ ID NO: 17;
   (b) a variable heavy (VH) chain sequence comprising the amino acid sequence of SEQ ID NO: 13 or a sequence having at least 90%, preferably at least 95% sequence identity to SEQ ID NO: 13; and
   a variable light (VL) chain sequence comprising the amino acid sequence of SEQ ID NO: 18 or a sequence having at least 90%, preferably at least 95% sequence identity to SEQ ID NO: 18;
   (c) a variable heavy (VH) chain sequence comprising the amino acid sequence of SEQ ID NO: 14 or a sequence having at least 90%, preferably at least 95% sequence identity to SEQ ID NO: 14; and
   a variable light (VL) chain sequence comprising the amino acid sequence of SEQ ID NO: 19 or a sequence having at least 90%, preferably at least 95% sequence identity to SEQ ID NO: 19;
   (d) a variable heavy (VH) chain sequence comprising the amino acid sequence of SEQ ID NO: 15 or a sequence having at least 90%, preferably at least 95% sequence identity to SEQ ID NO: 15; and
   a variable light (VL) chain sequence comprising the amino acid sequence of SEQ ID NO: 20 or a sequence having at least 90%, preferably at least 95% sequence identity to SEQ ID NO: 20;
   (e) a variable heavy (VH) chain sequence comprising the amino acid sequence of SEQ ID NO: 11 or a sequence having at least 90%, preferably at least 95% sequence identity to SEQ ID NO: 11; and
   a variable light (VL) chain sequence comprising the amino acid sequence of SEQ ID NO: 16 or a sequence having at least 90%, preferably at least 95% sequence identity to SEQ ID NO: 16.
8. The antibody, or antigen-binding fragment thereof, according to any one of embodiments 1 to 7, wherein the antibody, or antigen-binding fragment thereof, is a humanized antibody or a humanized antigen-binding fragment thereof.
9. The antibody, or antigen-binding fragment thereof, according to embodiment 8, wherein the antibody, or antigen-binding fragment thereof, is a deimmunized antibody or a deimmunized antigen-binding fragment thereof.
10. The antibody, or antigen-binding fragment thereof, according to embodiment 8 comprising at least one modified amino acid and/or defined amino acid selected from the group of HC 54S, HC 38R, HC 75K, HC 89T and LC 30S, defined by Chothia numbering scheme; and/or defined amino acid selected from the group of HC 55S HC 38R, HC 76K, HC 93T and LC 30S, defined by Kabat numbering scheme.
11. A polynucleotide encoding an antibody, or an antigen-binding fragment thereof, according to any one of embodiments 1 to 10.
12. A host cell comprising the polynucleotide of embodiment 11.
13. A method for producing an antibody comprising culturing the host cell of embodiment 12.
14. A pharmaceutical composition comprising the antibody, or antigen-binding fragment thereof, according to any one of embodiments 1 to 10, the polynucleotide of embodiment 11 or the host cell of embodiment 12, and a pharmaceutically acceptable carrier.
15. The pharmaceutical composition according to embodiment 14, comprising at least one further therapeutic agent.
16. The pharmaceutical composition according to embodiment 15, wherein the further therapeutic agent is selected from the group consisting of an anti-inflammatory agent, an immunomodulator, an antibiotic, an angiotensin-converting-enzyme inhibitor, a β-blocker and a diuretic.
17. The pharmaceutical composition according to embodiment 15 or 16, wherein the further therapeutic agent is
   (i) an anti-inflammatory agent selected from the group consisting of infliximab, adalimumab, certolizumab pegol, golimumab, etanercept, curcumin, IL-1RA, rilonacept, canakinumab, allopurinol, colchicine, prednisone, pentoxifylline, rosuvastatin and oxypurinol;
   (ii) an immunomodulator selected from the group consisting of antigenic peptide, immunoglobulin, methotrexate and stem cell-based therapy; or
   (iii) an antibiotic selected from the group consisting of anti-bacterial phages, rifaximin, vancomycin, and trimethoprim-sulfamethoxazole.
18. The antibody, or antigen-binding fragment thereof, of any one of embodiments 1 to 10, the polynucleotide of embodiment 11 or the host cell of embodiment 12, or the pharmaceutical composition of any one embodiments 14 to 18 for use in treating and/or preventing heart failure and/or an inflammatory cardiac disease.
19. The antibody, or antigen-binding fragment thereof, for use of embodiment 18, the polynucleotide for use of embodiment 18, or the host cell for use of embodiment 18, or the pharmaceutical composition for use of embodiment 18, wherein the inflammatory disease is an inflammatory disease of the heart, in particular inflammatory dilated cardiomyopathy, inflammatory cardiomyopathy, cardiomyopathy, inflammatory cardiomyopathy, myocarditis, pericarditis, perimyocarditis or myopericarditis.
20. The antibody, or antigen-binding fragment thereof, for use of embodiment 18, the polynucleotide for use of embodiment 18, or the host cell for use of embodiment 18, or the pharmaceutical composition for use of embodiment 18, wherein the inflammatory disease is reperfusion injury, allergy, asthma, coeliac disease, glomerulonephritis, hepatitis, inflammatory bowel disease or transplant rejection.
21. The antibody, or antigen-binding fragment thereof, of any one of embodiments 1 to 10, the polynucleotide of embodiment 11 or the host cell of embodiment 12, or the pharmaceutical composition of any one of embodiments 14 to 18 for use in the treatment and/or prevention of a disease or disorder associated with SARS-CoV-2 infection and/or SARS-CoV-2 vaccination.
22. The antibody, or antigen-binding fragment thereof, for use of embodiment 21, the polynucleotide for use of embodiment 21, or the host cell for use of embodiment 21, or the pharmaceutical composition for use of embodiment 21, wherein the disease or disorder associated with SARS-CoV-2 infection and/or SARS-CoV-2 vaccination is heart failure and/or an inflammatory disease of the heart.

Accordingly, the invention relates to an antibody, or an antigen-binding fragment thereof, pan-specifically binding to Gremlin-1 and Gremlin-2.

The term “antibody” is used herein in the broadest sense and encompasses various antibody structures, including but not limited to monoclonal antibodies, polyclonal antibodies, multispecific antibodies (e.g., bispecificantibodies), fully-human antibodies and antibody fragments so long as they exhibit the desired antigen-binding activity. Antibodies within the present invention may also be chimeric antibodies, recombinant antibodies, antigen-binding fragments of recombinant antibodies, humanized antibodies, recombinant human antibodies, heterologous antibodies, heterohybrid antibodies or antibodies displayed upon the surface of a phage or displayed upon the surface of a cell (e.g., a chimeric antigen receptor T cell).

The term “Gremlin-1”, as used herein, refers a protein encoded by the GREM1 gene. Gremlin-1 is a highly conserved protein with 184 amino acids mapped to chromosome 15q13.3 in humans and to chromosome 2 E4; 2 57.43 cM in mice. The amino acid sequences of human and murine Gremlin-1 are provided in GenBank as accession numbers AAZ29612.1 and AAH15293.1 and are referred herein as SEQ ID NO: 1. The sequence homology between human and murine Gremlin-1 is 98%.

The phrase “an antibody that specifically binds to Germlin-1”, as used herein, refers to an antibody or an antigen-binding fragment thereof that is capable of binding Germlin-1 with sufficient affinity such that the antibody or antigen-binding fragment thereof is useful as a preventive, diagnostic and/or therapeutic agent for the desired purpose disclosed herein, in particular for use in treating an inflammatory disease and/or heart failure.

In some embodiments, the antibody, or antigen-binding fragment thereof, of the invention, binds with a certain affinity to Gremlin-1, preferably to human Germlin-1 (SEQ ID NO: 1). In some embodiments, the antibody, or antigen-binding fragment thereof, of the invention shows a particularly low K_{d} for the binding to Gremlin-1.

The term “K_{d}”, as used herein, refers to the equilibrium dissociation constant of a particular antibody-antigen interaction. The skilled person is well-aware of various methods and assays suitable for determining the K_{d} of an antibody or antigen-binding fragment thereof as provided herein and as encompassed by the present invention.

In some embodiments, K_{d} is measured using surface plasmon resonance assays using a BIACORE®-2000 or a BIACORE @-3000 system (BIAcore, Inc., Piscataway, NJ), for example at 25° C. with immobilized antigen. For example, carboxymethylated dextran biosensor chips (CM5, BIACORE, Inc.) are activated with N-ethyl-N′-(3-dimethylaminopropyl) carbodiimide hydrochloride (EDC) and N-hydroxysuccinimide (NHS) according to the supplier's instructions. Antigen is diluted with 10 mM sodium acetate, pH 4.8, to 5 μg/ml (−0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of antibody are injected in PBS with 0.05% polysorbate 20 (TWEEN-20™) surfactant (PEST) at 25° C. at a flow rate of approximately 25 μl/min. Association rates (kₒₙ) and dissociation rates (k_{off}) are calculated using a simple one-to-one Langmuir binding model (BIACORE® T100 Evaluation Software) by simultaneously fitting the association and dissociation sensorgrams. The equilibrium dissociation constant (K_{d}) is calculated as the ratio k_{off}/kₒₙ. See, e.g., Chen et al., 1999, J. Mol. Biol. 293:865-881. If the on-rate exceeds 106 M-1 s-1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

The term “Gremlin-2” refers the protein encoded by the GREM2 gene, for example having an amino-acid sequence as shown by SEQ ID NO: 2.

Cross-specificity of an antibody, or antigen-binding fragment thereof, may be tested, for example, by assessing binding of the antibody or antigen-binding fragments thereof, under conventional conditions (see, e.g., Harlow and Lane, 1988 Antibodies: A Laboratory Manual, Cold Spring Harbor Laboratory Press, and Harlow and Lane, 1999 using Antibodies: A Laboratory Manual, Cold Spring Harbor Laboratory Press) to Gremlin-1 and Germlin-2. An antibody or antigen-binding fragment thereof showing specific binding to Gremlin-1 and Gremlin-2 is considered cross-specific. It is preferred, however, that the antibody or antigen-binding fragment thereof does not or does not essentially bind to any other measured (poly)peptide in order to be considered cross-specific for Gremlin-1 and Germlin-2.

These methods may comprise, inter alia, binding studies, blocking and competition studies with structurally and/or functionally closely related molecules. These binding studies also comprise FACS analysis, surface plasmon resonance, analytical ultracentrifugation, isothermal titration calorimetry, fluorescence anisotropy, fluorescence spectroscopy or by radiolabeled ligand binding assays. Cross-specificity can be determined experimentally by methods known in the art and methods as described herein. Such methods comprise, but are not limited to Western Blots, ELISA-, RIA-, ECL-, IRMA-tests and peptide scans.

The term “pan-specifically binding to Gremlin-1 and Gremlin-2”, as used herein, refers to cross-specificity, wherein, the cross-specificity is characterized in that a) between 20% and 200%, between 40 and 150%, between 45% and 130% or between 80% and 130% of the binding detected by a Gremlin-1 ELISA can be detected by a Gremlin-2 ELISA; and/or b) the EC₅₀ for the activity inhibition of Gremlin-2 is between 20% and 200%, between 40 and 150%, between 45% and 130% or between 80% and 130% of the EC₅₀ for the activity inhibition of Gremlin-1.

In certain embodiments, the invention relates to an antibody or antigen-binding fragment thereof, showing pan-specificity to Gremlin-1 and Gremlin-2, wherein, the cross-specificity is characterized in that between 20% and 200%, between 40 and 150%, between 45% and 130% or between 80% and 130% of the binding detected by the Gremlin-1 ELISA of Example 2 can be detected by the Gremlin-2 ELISA of Example 2.

In certain embodiments, the invention relates to an antibody or antigen-binding fragment thereof, showing pan-specificity to Gremlin-1 and Gremlin-2, wherein, the cross-specificity is characterized in that the EC₅₀ for the activity inhibition of Gremlin-2 in the in vitro Gremlin-2 neutralization assay is between 20% and 200%, between 40 and 150%, between 45% and 130% or between 80% and 130% of the EC₅₀ for the activity inhibition of Gremlin-1 in vitro Gremlin-1 neutralization assay.

The term “in vitro Gremlin-2 neutralization assay”, as used herein, refers to an assay, setup and conditions as described in the Example 3 “in vitro Gremlin-2 neutralization assay”. The assay detects ALP (alkaline phosphatase) activity in ATDC5 cells as a readout of Gremlin-2 mediated BMP4 activity inhibition.

In certain embodiments, the invention relates to an antibody or antigen-binding fragment thereof, showing pan-specificity to Gremlin-1 and Gremlin-2, wherein, the cross-specificity is characterized in that the EC₅₀ for the activity inhibition of Gremlin-2 in the in vitro Gremlin-2 neutralization assay lower than the activity inhibition of Gremlin-1 in vitro Gremlin-1 neutralization assay.

Antibodies that show pan-specificity to Gremlin-1 and Gremlin-2 can achieve a particularly pronounced effect as described e.g. Ex. 4.

That is, the present invention is based, at least in part, on the surprising discovery that an antibody or an antigen-binding fragment thereof, that pan-specifically binds to Gremlin-1 and Gremlin-2 enables particular uses and biologic effects, such as the anti-inflammatory and cardioprotective effects described herein.

In certain embodiments, the invention relates to the antibody, or antigen-binding fragment thereof, according to the invention, wherein the antibody or antigen-binding fragment thereof, binds to Gremlin-2 within the amino acid sequence SEQ ID NO: 2.

In certain embodiments, the invention relates to the antibody, or antigen-binding fragment thereof, according to the invention, wherein the antibody or antigen-binding fragment thereof, binds to Gremlin-1 within the amino acid sequence SEQ ID NO: 1.

In certain embodiments, the invention relates to the antibody, or antigen-binding fragment thereof, according to the invention, wherein the antibody or antigen-binding fragment thereof, binds to Gremlin-1 within the amino acid sequence SEQ ID NO: 1 and to Gremlin-2 within the amino acid sequence SEQ ID NO: 2.

An antibody or fragment that “binds to an epitope” within a defined region of a protein is an antibody or fragment that requires the presence of one or more of the amino acids within that region for binding to the protein.

In certain embodiments, an antibody or antigen-binding fragment that “binds to an epitope” within a defined region of a protein is identified by mutation analysis, in which amino acids of the protein are mutated, and binding of the antibody to the resulting altered protein (e.g., an altered protein comprising the epitope) is determined to be at least 20% of the binding to unaltered protein. In some embodiments, an antibody or antigen-binding fragment “that binds to an epitope” within a defined region of a protein is identified by mutation analysis, in which amino acids of the protein are mutated, and binding of the antibody to the resulting altered protein (e.g., an altered protein comprising the epitope) is determined to be at least 30%, at least 40%, at least 50%, at least 60%, at least 70%, at least 80%, or at least 90% of the binding to unaltered protein. In certain embodiments, binding of the antibody or antigen-binding fragment is determined by FACS, WB or by a suitable binding assay such as ELISA.

Thus, the antibody or antigen-binding fragment may be any antibody or antigen-binding fragment which specifically binds to/specifically recognizes/interacts with an epitope within the amino acid sequences of SEQ ID NO: 35 and/or SEQ ID NO: 36. Accordingly, the invention also provides antibodies binding to the same epitope as any of the specific antibodies provided herein.

The term “binding to” as used in the context of the present invention defines a binding (interaction) of at least two “antigen-interaction-sites” with each other. The term “antigen-interaction-site” defines, in accordance with the present invention, a motif of a polypeptide, i.e., a part of the antibody or antigen-binding fragment of the present invention, which shows the capacity of specific interaction with a specific antigen or a specific group of antigens of Gremlin-1 and/or Gremlin-2. Said binding/interaction is also understood to define a “specific recognition”. The term “specifically recognizing” means in accordance with this invention that the antibody is capable of specifically interacting with and/or binding to at least two amino acids of Gremlin-1 and Gremlin-2 as defined herein, in particular interacting with/binding to at least 2, at least 3, at least 4, at least 6, at least 7, at least 8, at least 9, at least 10, at least 11, at least 12, at least 13, at least 14, at least 15, at least 16 or all amino acids within the amino acid sequences of SEQ ID NO: 35 and/or SEQ ID NO: 36.

The inventors found that binding to Gremlin-1 within the amino acid sequence SEQ ID NO: 35 and/or to Gremlin-2 within the amino acid sequence SEQ ID NO: 36 enables antibodies or antigen binding fragments to be pan-specific and/or to exhibit a particularly pronounced biologic effect on Gremlin-1 and/or Gremlin-2 activity (see e.g. Ex. 4).

Accordingly, the present invention is based, at least in part, on the surprising discovery that an antibody or an antigen-binding fragment thereof, that binds to Gremlin-1 within the amino acid sequence SEQ ID NO: 35 and/or to Gremlin-2 within the amino acid sequence SEQ ID NO: 36 enables particular uses and biologic effects, such as the anti-inflammatory and cardioprotective effects described herein.

In certain embodiments, the invention relates to the antibody, or antigen-binding fragment thereof, according to the invention, comprising at least one CDR as defined by the SEQ ID NO: 4.

In certain embodiments, the invention relates to the antibody, or antigen-binding fragment thereof, according to the invention, comprising at least one CDR as defined by the SEQ ID NO: 10.

In certain embodiments, the invention relates to the antibody, or antigen-binding fragment thereof, according to the invention, comprising a variable heavy (VH) chain comprising CDR1 as defined in SEQ ID NO: 3, CDR2 as defined in SEQ ID NO: 4 and CDR3 as defined in SEQ ID NO: 5 and a variable light (VL) chain comprising CDR1 as defined in SEQ ID NO: 6, CDR2 is GAT and CDR3 as defined in SEQ ID NO: 8; In certain embodiments, the invention relates to the antibody, or antigen-binding fragment thereof, according to the invention, comprising a variable heavy (VH) chain comprising CDR1 as defined in SEQ ID NO: 3, CDR2 as defined in SEQ ID NO: 9 and CDR3 as defined in SEQ ID NO: 5 and a variable light (VL) chain comprising CDR1 as defined in SEQ ID NO: 10, CDR2 is GAT and CDR3 as defined in SEQ ID NO: 8.

In certain embodiments, the invention relates to the antibody, or antigen-binding fragment thereof, according to the invention, comprising a variable heavy (VH) chain comprising CDR1 as defined in SEQ ID NO: 3, CDR2 as defined in SEQ ID NO: 4 and CDR3 as defined in SEQ ID NO: 5 and a variable light (VL) chain comprising CDR1 as defined in SEQ ID NO: 10, CDR2 is GAT and CDR3 as defined in SEQ ID NO: 8.

The term “CDR”, as used herein, relates to “complementary determining region”, which is well known in the art. The CDRs are parts of immunoglobulins that determine the specificity of said molecules and make contact with a specific ligand. The CDRs are the most variable part of the molecule and contribute to the diversity of these molecules. There are three CDR regions CDR1, CDR2 and CDR3 in each V domain. CDR-H depicts a CDR region of a variable heavy chain and CDR-L relates to a CDR region of a variable light chain. VH means the variable heavy chain and VL means the variable light chain. The CDR regions of an Ig-derived region may be determined as described in Kabat et al., 1991, 5th edn. US Department of Health and Human Services, Public Health Service, NIH.; Chothia, 1987, J. Mol. Biol. 196, 901-917; Chothia, 1989 Nature 342, 877-883.

Antibodies comprising the above-mentioned CDRs have proven useful in the context of the invention (see e.g. Examples). As such, the inventors provide means and methods for removal of potential sequence liabilities and de-immunization of pan specific huGREM1/2 antibodies while maintaining and/or improving neutralization activities.

Accordingly, the antibody, or the antigen-binding fragment thereof, of the invention, that comprise one or more of the above mentioned CDRs are surprisingly useful in the context of the invention and/or uses described herein.

In certain embodiments, the invention relates to the antibody, or antigen-binding fragment thereof, according to the invention, wherein the antibody or the antigen-binding fragment thereof comprises a variable heavy (VH) chain sequence comprising the amino acid sequence of SEQ ID NO: 12 or a sequence having at least 90%, preferably at least 95% sequence identity to SEQ ID NO: 12; and a variable light (VL) chain sequence comprising the amino acid sequence of SEQ ID NO: 17 or a sequence having at least 90%, preferably at least 95% sequence identity to SEQ ID NO: 17;

In certain embodiments, the invention relates to the antibody, or antigen-binding fragment thereof, according to the invention, wherein the antibody or the antigen-binding fragment thereof comprises a variable heavy (VH) chain sequence comprising the amino acid sequence of SEQ ID NO: 13 or a sequence having at least 90%, preferably at least 95% sequence identity to SEQ ID NO: 13; and a variable light (VL) chain sequence comprising the amino acid sequence of SEQ ID NO: 18 or a sequence having at least 90%, preferably at least 95% sequence identity to SEQ ID NO: 18;

In certain embodiments, the invention relates to the antibody, or antigen-binding fragment thereof, according to the invention, wherein the antibody or the antigen-binding fragment thereof comprises a variable heavy (VH) chain sequence comprising the amino acid sequence of SEQ ID NO: 14 or a sequence having at least 90%, preferably at least 95% sequence identity to SEQ ID NO: 14; and a variable light (VL) chain sequence comprising the amino acid sequence of SEQ ID NO: 19 or a sequence having at least 90%, preferably at least 95% sequence identity to SEQ ID NO: 19;

In certain embodiments, the invention relates to the antibody, or antigen-binding fragment thereof, according to the invention, wherein the antibody or the antigen-binding fragment thereof comprises a variable heavy (VH) chain sequence comprising the amino acid sequence of SEQ ID NO: 15 or a sequence having at least 90%, preferably at least 95% sequence identity to SEQ ID NO: 15; and a variable light (VL) chain sequence comprising the amino acid sequence of SEQ ID NO: 20 or a sequence having at least 90%, preferably at least 95% sequence identity to SEQ ID NO: 20;

In certain embodiments, the invention relates to the antibody, or antigen-binding fragment thereof, according to the invention, wherein the antibody or the antigen-binding fragment thereof comprises a variable heavy (VH) chain sequence comprising the amino acid sequence of SEQ ID NO: 11 or a sequence having at least 90%, preferably at least 95% sequence identity to SEQ ID NO: 11; and a variable light (VL) chain sequence comprising the amino acid sequence of SEQ ID NO: 16 or a sequence having at least 90%, preferably at least 95% sequence identity to SEQ ID NO: 16.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

In some embodiments, the antibody or fragment thereof, pan-specifically binding to Gremlin-1 and Gremlin-2 comprises a heavy chain variable domain (VH) sequence having at least 85%, 86%, 87%, 88%, 89%, 90%, 91%, 92%, 93%, 94%, 95%, 96%, 97%, 98%, 99%, or 100% sequence identity to the amino acid sequence of SEQ ID NO: 11, SEQ ID NO: 12, SEQ ID NO: 13, SEQ ID NO: 14 or SEQ ID NO: 15. In certain embodiments, a VH sequence having at least 85%, 86%, 87%, 88%, 89%, 90%, 91%, 92%, 93%, 94%, 95%, 96%, 97%, 98%, or 99% identity to the amino acid sequence of SEQ ID NO: 11, SEQ ID NO: 12, SEQ ID NO: 13, SEQ ID NO: 14 or SEQ ID NO: 15 contains substitutions, insertions, or deletions relative to the reference sequence, but the antibody or fragment thereof, comprising that sequence retains the ability to bind to pan-specifically binding to Gremlin-1 and Gremlin-2. In certain embodiments, a total of 1 to 10 amino acids have been substituted, inserted and/or deleted in SEQ ID NO: 11, SEQ ID NO: 12, SEQ ID NO: 13, SEQ ID NO: 14 or SEQ ID NO: 15. In certain embodiments, a total of 1 to 6 amino acids have been substituted, inserted and/or deleted in SEQ ID NO: 11, SEQ ID NO: 12, SEQ ID NO: 13, SEQ ID NO: 14 or SEQ ID NO: 15. In certain embodiments, substitutions, insertions, or deletions occur in regions outside the CDRs (i.e., in the FRs). In a preferred embodiment, a total of 1 to 6 amino acids in SEQ ID NO: 11, SEQ ID NO: 12, SEQ ID NO: 13, SEQ ID NO: 14 or SEQ ID NO: 15 have been substituted to optimize the expression in mammalian cells. In certain embodiments, substitutions, insertions, or deletions occur in regions outside the CDRs (i.e., in the FRs). Optionally, the antibody or fragment thereof, pan-specifically binding to Gremlin-1 and Gremlin-2 comprises the VH sequence of SEQ ID NO: 11, SEQ ID NO: 12, SEQ ID NO: 13, SEQ ID NO: 14 or SEQ ID NO: 15, including post-translational modifications of that sequence.

In some embodiments, the antibody or fragment thereof, pan-specifically binding to Gremlin-1 and Gremlin-2 comprises a heavy chain variable domain (VL) sequence having at least 85%, 86%, 87%, 88%, 89%, 90%, 91%, 92%, 93%, 94%, 95%, 96%, 97%, 98%, 99%, or 100% sequence identity to the amino acid sequence of SEQ ID NO: 16, SEQ ID NO: 17, SEQ ID NO: 18, SEQ ID NO: 19 or SEQ ID NO: 20. In certain embodiments, a VL sequence having at least 85%, 86%, 87%, 88%, 89%, 90%, 91%, 92%, 93%, 94%, 95%, 96%, 97%, 98%, or 99% identity to the amino acid sequence of SEQ ID NO: 16, SEQ ID NO: 17, SEQ ID NO: 18, SEQ ID NO: 19 or SEQ ID NO: 20 contains substitutions, insertions, or deletions relative to the reference sequence, but the antibody or fragment thereof, comprising that sequence retains the ability to bind to pan-specifically binding to Gremlin-1 and Gremlin-2. In certain embodiments, a total of 1 to 10 amino acids have been substituted, inserted and/or deleted in SEQ ID NO: 16, SEQ ID NO: 17, SEQ ID NO: 18, SEQ ID NO: 19 or SEQ ID NO: 20. In certain embodiments, a total of 1 to 6 amino acids have been substituted, inserted and/or deleted in SEQ ID NO: 16, SEQ ID NO: 17, SEQ ID NO: 18, SEQ ID NO: 19 or SEQ ID NO: 20. In certain embodiments, substitutions, insertions, or deletions occur in regions outside the CDRs (i.e., in the FRs). In a preferred embodiment, a total of 1 to 6 amino acids in SEQ ID NO: 16, SEQ ID NO: 17, SEQ ID NO: 18, SEQ ID NO: 19 or SEQ ID NO: 20 have been substituted to optimize the expression in mammalian cells. In certain embodiments, substitutions, insertions, or deletions occur in regions outside the CDRs (i.e., in the FRs). Optionally, the antibody or fragment thereof, pan-specifically binding to Gremlin-1 and Gremlin-2 comprises the VL sequence of SEQ ID NO: 16, SEQ ID NO: 17, SEQ ID NO: 18, SEQ ID NO: 19 or SEQ ID NO: 20, including post-translational modifications of that sequence. In certain embodiments, the substitutions described herein comprise an asparagine to serine mutation.

Antibodies comprising the above-mentioned VL and VH sequences have proven useful in the context of the invention (see e.g. Examples).

Accordingly, the antibody, or the antigen-binding fragment thereof, of the invention, that comprise one or more of the above mentioned VL and VH sequences are surprisingly useful in the context of the invention and/or uses described herein.

In certain embodiments, the invention relates to the antibody, or antigen-binding fragment thereof, according to the invention, wherein the antibody, or antigen-binding fragment thereof, is a humanized antibody or a humanized antigen-binding fragment thereof.

In certain embodiments, the invention relates to the antibody, or antigen-binding fragment thereof, according to the invention, wherein the antibody, or antigen-binding fragment thereof, is a deimmunized antibody or a deimmunized antigen-binding fragment thereof.

In certain embodiments, antibody variants having one or more amino acid substitutions are provided. Amino acid substitutions may be introduced into an antibody of interest and the products screened for a desired activity, e.g., retained/improved antigen binding, decreased immunogenicity, or altered ADCC or CDC.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, increased Gremlin-1 activity reduction capacity, reduced immunogenicity) relative to the parent antibody and/or will have substantially retained certain biological properties of the parent antibody. An exemplary substitutional variant is an affinity-matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity or Gremlin-1 activity reduction capacity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, 2008, Methods Mol. Biol. 207:179-196), and/or SDRs (a-CDRs), with the resulting variant VH or VL being tested for binding affinity. Affinity maturation by constructing and reselecting from secondary libraries has been described, e.g., in Hoogenboom et al., 2002 in Methods in Molecular Biology 178:1-37. In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted. In another embodiment look-through mutagenesis is used to optimize antibody affinity with a multidimensional mutagenesis method that simultaneously assesses and optimizes combinatorial mutations of selected amino acids (Rajpal, Arvind et al., 2005, Proceedings of the National Academy of Sciences of the United States of America vol. 102, 24:8466-71).

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity and/or Gremlin-1 activity reduction capacity may be made in CDRs. Such alterations may be outside of CDR “hotspots” or SDRs. In certain embodiments of the variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells, 1989, Science, 244: 1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighboring residues may be targeted or eliminated as candidates for substitution. Variants may be screened to determine whether they contain the desired properties.

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al., 1997, TIBTECH 15:26-32. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g., complex, hybrid and high mannose structures) as measured by MALDI-TOF mass spectrometry, as described in WO 2008/077546, for example. Asn297 refers to the asparagine residue located at about position 297 in the Fc region (Eu numbering of Fe region residues); however, Asn297 may also be located about ±3 amino acids upstream or downstream of position 297, i.e., between positions 294 and 300, due to minor sequence variations in antibodies. Such fucosylation variants may have an altered influence on inflammation (Irvine, Edward B, and Galit Alter., 2020, Glycobiology vol. 30, 4: 241-253). See, e.g., US 2003/0157108; US 2004/0093621. Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO 2005/053742; WO 2002/031140; Okazaki et al. 2004 J. Mol. Biol. 336:1239-1249; Yamane-Ohnuki et al., 2004, Biotech. Bioeng. 87: 614. Examples of cell lines capable of producing defucosylated antibodies include Lec13 CHO cells deficient in protein fucosylation (Ripka et al., 1986, Arch. Biochem. Biophys. 249:533-545; US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al., 2004, Biotech. Bioeng. 87: 614; Kanda, Y. et al., 2006, Biotechnol. Bioeng., 94(4):680-688; and WO 2003/085107).

Antibodies variants are further provided with bisected oligosaccharides, e.g., in which a biantennary oligosaccharide attached to the Fc region of the antibody is bisected by GlcNAc. Such antibody variants may have altered fucosylation and/or altered influence on inflammation (Irvine, Edward B, and Galit Alter., 2020, Glycobiology vol. 30, 4: 241-253). Examples of such antibody variants are described, e.g., in WO 2003/011878; U.S. Pat. No. 6,602,684; and US 2005/0123546. Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

Antibodies with increased half-lives and improved binding to the neonatal Fc receptor (FcRn), which is responsible for the transfer of maternal IgGs to the fetus (Guyer et al., 1976, J. Immunol. 117:587 and Kirn et al., 1994 J. Immunol. 24:249), are described in US2005/0014934. Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (US 2006/0194291).

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

In certain embodiments, an antibody provided herein may be further modified to contain additional non-proteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In certain embodiments, the invention relates to the antibody, or antigen-binding fragment thereof, according to the invention wherein the antibody is de-amidated.

In certain embodiments, the invention relates to the antibody, or antigen-binding fragment thereof, according to the invention wherein the antibody comprises at least one iso-Asp.

In certain embodiments, the invention relates to the antibody, or antigen-binding fragment thereof, according to the invention wherein the antibody comprises an asparagine to serine mutation, e.g., to prevent deamination and increase half-life. In certain embodiments, the invention relates to the antibody, or antigen-binding fragment thereof, according to the invention comprising at least one defined amino acid selected from the group of HC 54S, HC 38R, HC 75K, HC 89T and LC 30S.

The defined amino acids described herein are numbered according to the Chothia numbering scheme.

In certain embodiments, the invention relates to the antibody, or antigen-binding fragment thereof, according to the invention comprising at least one defined amino acid selected from the group of HC 55S, HC 38R, HC 76K, HC 93T, and LC 30S.

The defined amino acids described herein are numbered according to the Kabat numbering scheme.

In certain embodiments, the invention relates to the antibody, or antigen-binding fragment thereof, according to the invention comprising at least one modified amino acid and defined amino acid selected from the group of HC 54S, HC 38R, HC 75K, HC 89T, LC 30S, defined by the Chothia numbering scheme.

In certain embodiments, the invention relates to the antibody, or antigen-binding fragment thereof, according to the invention comprising at least one modified amino acid and defined amino acid selected from the group of HC 55S, HC 38R, HC 76K, HC 93T, and LC 30S, defined by the Kabat numbering scheme.

The inventors found, that certain mutations do not or not substantially reduce the binding affinity to Gremlin-1 and Gremlin-2, while improving stability, deimmunization and/or the pharmacokinetic of the antibody or the fragment thereof. In particular, the N55S (Kabat numbering) mutation was found to increase in vivo half-life of the antibodies Var_5 and Var_7.

Furthermore, the inventors found, that certain variations (e.g. Var_5 and Var_7) possess superior neutralization activity against huGREM1 when compared to the chimeric antibody control.

Accordingly, the invention is at least in part based on the surprising finding that certain mutations improve the drug properties of the antibody or fragment thereof, described herein.

In certain embodiments, the invention relates to an antibody, or antigen-binding fragment thereof, for use according to the invention, wherein the binding of the antibody, or the antigen-binding fragment thereof, to Gremlin-1 reduces Gremlin-1 activity to inhibit the activity of BMP.

The term “bone morphogenetic protein” or “BMP”, as used herein, relates to tissue cytokines belonging to the TGF-β superfamily. BMPs have been originally discovered by their capability to induce bone and cartilage formation, but they play an essential role as morphogens during early embryonic development and are essential in organ homeostasis. BMPs, which are named according to their potential to induce bone and cartilage development (Wozney, J. M. et al., 1988, Science 242, 1528-1534), it is surprising that altered BMP activity is beneficial for use in treating and/or preventing a disease, disorder and/or condition described herein, that are not linked to bone and cartilage development.

As described herein, antibodies of the invention can particularly efficiently reduce Germlin-1 mediated BMP inhibition (e.g., Example 3) and enabling beneficial effects for use in treating and/or preventing the diseases, disorders and/or conditions described herein (e.g., Ex. 4).

In some embodiments, the antibody, or the antigen-binding fragment, of the invention reduces the Gremlin-1 mediated BMP inhibition in one or more assays more than antibodies of the prior art that specifically bind to Gremlin-1.

Therefore, the antibody, or the antigen-binding fragment thereof, of the invention can indirectly increase activity of BMP, which results in beneficial effects for use in treating and/or preventing the diseases, disorders and/or conditions described herein.

Accordingly, the antibody, or the antigen-binding fragment thereof, of the invention, wherein the binding of the antibody, or the antigen-binding fragment thereof, to Gremlin-1 inhibits Gremlin-1 activity to inhibit the activity of BMP are surprisingly useful for use in treating and/or preventing the diseases, disorders and/or conditions described herein.

In certain embodiments, the invention relates to an antibody, or antigen-binding fragment thereof, for use according to the invention, wherein the binding of the antibody, or the antigen-binding fragment thereof, to Gremlin-1 inhibits Gremlin-1 binding to BMP2, and/or BMP7.

BMPs are the largest group in the TGF-β superfamily play an essential role as morphogens during early embryonic development (Bier, E., and De Robertis, E. M., 2015, Science 348, aaa5838), with BMP2 and BMP4 being critical factors for cardiac development (Jiao et al., 2003; Ma et al., 2005). Moreover, they regulate the homeostasis and functional preservation of the cardiac tissue (Hanna, A., and Frangogiannis, N. G., 2019, Front Cardiovasc Med 6, 140.) and are involved in the remodeling process of the injured heart (Rutkovskiy, A. et al., 2017, Scand J Clin Lab Invest 77, 321-331; Wu, X. et al., 2014, Life Sci 97, 145-154). Mature BMPs are secreted to the extracellular space and form extracellular matrix-associated dimers before they bind to BMP receptor types 1 and 2.

In some embodiments the antibody, or antigen-binding fragment thereof, of the invention inhibits Gremlin-1 binding to BMP2. In some embodiments, the antibody, or the antigen-binding fragment, of the invention reduces the Gremlin-1 mediated BMP2 inhibition in one or more assays more than antibodies of the prior art that specifically bind to Gremlin-1

In some embodiments the antibody, or antigen-binding fragment thereof, of the invention inhibits Gremlin-1 binding to BMP7. In some embodiments, the antibody, or the antigen-binding fragment, of the invention reduces the Gremlin-1 mediated BMP7 inhibition in one or more assays more than antibodies of the prior art that specifically bind to Gremlin-1.

As described herein, antibodies of the invention can particularly efficiently reduce Germlin-1 mediated BMP4 inhibition (e.g., Ex. 3) and enabling beneficial effects for use in treating and/or preventing the diseases, disorders and/or conditions described herein (e.g., Ex. 4). In some embodiments the antibody, or antigen-binding fragment thereof, of the invention inhibits Gremlin-1 binding to BMP2 to a similar extent compared to BMP4. In some embodiments the antibody, or antigen-binding fragment thereof, of the invention inhibits Gremlin-1 binding to BMP7 to a similar extent compared to BMP4. Therefore, the antibody, or the antigen-binding fragment thereof, of the invention can indirectly increase activity of BMP2, and BMP7, which results in beneficial effects for use in treating and/or preventing the diseases, disorders and/or conditions described herein.

Accordingly, the antibody, or the antigen-binding fragment thereof, of the invention, wherein the binding of the antibody, or the antigen-binding fragment thereof, to Gremlin-1 inhibits Gremlin-1 activity to inhibit the activity of BMP2, and/or BMP7 are surprisingly useful for use in treating and/or preventing the diseases, disorders and/or conditions described herein.

In any of the embodiments described herein, the antibody may be a monoclonal antibody. In any of the embodiments described herein, the antibody may be human, humanized, or chimeric antibody. There are five major classes of antibodies: IgA, IgD, IgE, IgG, and IgM, and several of these may be further divided into subclasses, e.g., IgG1, IgG2, IgG3, IgG4, IgA1, and IgA2. The heavy chain constant domains that correspond to the different classes of immunoglobulins are called α, δ, ε, γ, and μ, respectively. In any of the embodiments described herein, the antibody may be an antibody specifically binding to Gremlin-1. In any of the embodiments described herein, the antibody may be an IgG1, IgG2a or IgG2b, IgG3, IgG4, IgM, IgA (e.g., IgA1, IgA2), IgAsec, IgD, IgE. The antibodies can be full length or can include only an antigen-binding fragment such as the antibody constant and/or variable domain of IgG1, IgG2, IgG3, IgG4, IgM, IgA1, IgA2, IgAsec, IgD or IgE or could consist of a Fab fragment, an F(ab′) fragment, an Fv fragment, an F(ab′)2 fragment and/or a single-chain Fv fragment.

A “Fab fragment” as used herein is comprised of one light chain and the CH1 and variable regions of one heavy chain. The heavy chain of a Fab molecule cannot form a disulfide bond with another heavy chain molecule.

A “F(ab′) fragment” contains one light chain and a portion of one heavy chain that contains the VH domain and the C H1 domain and also the region between the CH1 and C H2 domains, such that an interchain disulfide bond can be formed between the two heavy chains of two F(ab′) fragments to form a F(ab′) 2 molecule.

The “Fv fragment” comprises the variable regions from both the heavy and light chains, but lacks the constant regions.

A “F(ab′)2 fragment” contains two light chains and two heavy chains containing a portion of the constant region between the CH1 and CH2 domains, such that an interchain disulfide bond is formed between the two heavy chains. A F(ab′)2 fragment thus is composed of two Fab′ fragments that are held together by a disulfide bond between the two heavy chains.

“Single-chain Fv” or “scFv” antibody fragments have, in the context of the invention, the VH and VL domains of an antibody, wherein these domains are present in a single polypeptide chain. Generally, the scFv polypeptide further comprises a polypeptide linker between the VH and VL domains which enables the scFv to form the desired structure for antigen binding. Techniques described for the production of single chain antibodies are described, e.g., in Pluckthun, A., 1994, The Pharmacology of Monoclonal Antibodies, 269-315.

An “Fc region” contains two heavy chain fragments comprising the CH2 and CH3 domains of an antibody. The two heavy chain fragments are held together by two or more disulfide bonds and by hydrophobic interactions of the CH3 domains.

Antibodies, antibody constructs, antibody fragments, antibody derivatives (all being Ig-derived) to be employed in accordance with the invention or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook et al., 1989, Molecular Cloning: A Laboratory Manual; Cold Spring Harbor Laboratory Press. The term “Ig-derived domain” particularly relates to (poly) peptide constructs comprising at least one CDR. Fragments or derivatives of the recited Ig-derived domains define (poly) peptides which are parts of the above antibody molecules and/or which are modified by chemical/biochemical or molecular biological methods. Corresponding methods are known in the art and described inter alia in laboratory manuals (see Sambrook et al., Molecular Cloning: A Laboratory Manual; Cold Spring Harbor Laboratory Press, 2nd edition (1989) and 3rd edition (2001); Gerhardt et al., 1994, Methods for General and Molecular Bacteriology ASM Press; Lefkovits, 1997, Immunology Methods Manual: The Comprehensive Sourcebook of Techniques; Academic Press); Golemis, 2002, Protein-Protein Interactions: A Molecular Cloning Manual Cold Spring Harbor Laboratory Press).

In certain embodiments the antibody, or antigen binding fragment thereof, of the invention is of a certain class or a certain fragment described above to enable a certain distribution in the body. For example, the antibody, or antigen binding fragment thereof, of the invention may be of the a certain class (e.g. IgM, IgA, IgAsec, IgD, IgE), or a fragment as described above in order to avoid active placenta transfer and/or accumulation in the fetus during pregnancy (e.g. as described for IgG antibodies, e.g., in Palmeira, Patricia, et al. 2012 Clinical and Developmental Immunology).

Accordingly, in the context of the present invention, the antibody molecule described herein above is selected from the group consisting of a full antibody (immunoglobulin, like an IgG1, an IgG2, an IgG2a, an IgG2b, an IgA1, an IgGA2, an IgG3, an IgG4, an IgA, an IgM, an IgD or an IgE), F(ab)-, Fab′-SH-, Fv-, Fab′-, F(ab′)2-fragment, a chimeric antibody, a CDR-grafted antibody, a fully human antibody, a bivalent antibody-construct, an antibody-fusion protein, a synthetic antibody, bivalent single chain antibody, a trivalent single chain antibody and a multivalent single chain antibody.

In some embodiments, the invention relates to a method of detecting Gremlin-1 and Gremlin-2 in a biological sample comprising contacting the biological sample with an antibody, of the invention that is cross-specific for Gremlin-1 and Gremlin-2 under conditions permissive for binding of the antibody to Gremlin-1 and Gremlin-2, and detecting whether a complex is formed between the antibody and Gremlin-1 and Gremlin-2 in the biological sample.

The cross-specificity of the antibody of the invention for Gremlin-1 and Gremlin-2 a surprisingly efficient and/or accurate detection of Gremlin-1 and 2.

In some embodiments, the invention relates to a method of detecting Gremlin-1 in a biological sample comprising contacting the biological sample with the antibody of the invention under conditions permissive for binding of the antibody to Gremlin-1, and detecting whether a complex is formed between the antibody and Gremlin-1 in the biological sample.

As described herein an antibody of the invention can detect particularly low concentrations of human Gremlin-1 (huGREM1) (Ex. 3).

In some embodiments, the invention relates to a method for quantifying the concentration of Gremlin-2 in a sample, the method comprising the steps of:
a) quantifying the concentration of Gremlin-1 and Gremlin-2 in a biological sample comprising contacting the biological sample with an antibody, of the invention that is cross-specific for Gremlin-1 and Gremlin-2 under conditions permissive for binding of the antibody to Gremlin-1 and Gremlin-2, and detecting whether a complex is formed between the antibody and Gremlin-1 and Gremlin-2 in the biological sample.
b) quantifying the concentration of Gremlin-1 in a biological sample comprising contacting the biological sample with an antibody that specifically binds to Gremlin 1 but not to Gremlin-2 under conditions permissive for binding of the antibody to Gremlin-1, and quantifying the concentration of Gremlin-1 based on complex formation between the antibody and Gremlin-1 in the biological sample.
c) quantifying the concentration of Gremlin-2 in the sample, wherein quantifying the concentration of Gremlin-2 in the sample involves subtracting the concentration of Gremlin-1 in the sample from the total concentration of Gremlin-1 and Gremlin-2 in the sample.

The cross-specificity for Gremlin-1 and Gremlin-2 combined with a high affinity and/or high Gremlin-1 activity reduction capacity of the antibodies of the invention for Gremlin-1 enable a surprisingly efficient and/or accurate detection of Gremlin 2.

Patients with heart failure, which have adverse outcome, have a positive correlation between the amount of Gremlin-1, cardiac fibrosis and the degree of cardiac dysfunction in heart failure patients (Mueller K A et al., 2013, J Card Fail, 19(10):678-84).

In some embodiments, the invention relates to a method predicting and/or diagnosing heart failure comprising the steps of:
(a) quantifying the concentration of Gremlin-1 in a biological sample comprising contacting the biological sample with the antibody of the invention under conditions permissive for binding of the antibody to Gremlin-1, and quantifying the concentration of Gremlin-1 based on complex formation between the antibody and Gremlin-1 in the biological sample.
(b) comparing the concentration of Gremlin-1 that has been determined in step (a) to a reference value; and
(c) predicting and/or diagnosing heart failure in said subject based on the comparison made in step (b).

In some embodiments, the invention relates to a method for determining whether a subject is susceptible to the treatment of heart failure comprising the steps of:
(a) quantifying the concentration of Gremlin-1 in two or more biological samples in two or more samples that have been obtained from said subject at an earlier and a later time point comprising contacting the biological sample with the antibody of the invention under conditions permissive for binding of the antibody to Gremlin-1, and quantifying the concentration of Gremlin-1 based on complex formation between the antibody and Gremlin-1 in the biological sample.
(b) comparing the quantifications of Gremlin-1 that have been determined in step (a) to one or more reference values; and
(c) determining that said subject is susceptible to the treatment of heart failure.

In embodiments, the antibody, or antigen binding fragment thereof, of the invention can include (e.g., be fused to or coupled to) one or more labels (e.g., detectable labels). A label can be, without limitation, a fluorescent label (e.g., a fluorophore), a radioactive label, or an enzyme. Examples of detectable labels include, without limitation, R-Phycoerythrin (PE), CTO, GFP, fluorogen-activating protein (FAP), Gaussia Luciferase (GLuc), Cypridina Luciferase (Clue), radionuclides, and biotin.

In some embodiments, any antibody, or antigen binding fragment thereof, of the invention is used in any method described herein.

In certain embodiments, the invention relates to a polynucleotide encoding an antibody, or an antigen-binding fragment thereof, according to the invention.

The term “polynucleotide”, as used herein, refers to a nucleic acid sequence. The nucleic acid sequence may be a DNA or a RNA sequence, preferably the nucleic acid sequence is a DNA sequence. The polynucleotides of the present invention either essentially consist of the aforementioned nucleic acid sequences or comprise the aforementioned nucleic acid sequences. Thus, they may contain further nucleic acid sequences as well. The polynucleotides of the present invention shall be provided, preferably, either as an isolated polynucleotide (i.e. isolated from its natural context) or in genetically modified form. An isolated polynucleotide as referred to herein also encompasses polynucleotides which are present in cellular context other than their natural cellular context, i.e. heterologous polynucleotides. The term polynucleotide encompasses single as well as double stranded polynucleotides. Moreover, comprised are also chemically modified polynucleotides including naturally occurring modified polynucleotides such as glycosylated or methylated polynucleotides or artificial modified one such as biotinylated polynucleotides.

In an embodiment, the polynucleotide of the invention encodes at least one of a variable heavy (VH) chain sequence and/or a variable light (VL) chain sequence of an antibody that specifically binds to Gremlin-1.

In certain embodiments the polynucleotide encoding an antibody, or an antigen-binding fragment thereof, of the invention is suitable for the use as a vector.

In certain embodiments the polynucleotide encoding an antibody, or an antigen-binding fragment thereof, of the invention is suitable for the use as a vector for transient transfection.

In certain embodiments the polynucleotide encoding an antibody, or an antigen-binding fragment thereof, of the invention is suitable for the use as a vector for stable transfection.

In certain embodiments the polynucleotide encoding an antibody, or an antigen-binding fragment thereof, of the invention is suitable for the use as a vector that enables production of the antibody, or antigen-binding fragment thereof, in a host cell.

In certain embodiments, the invention relates to a polynucleotide comprising a nucleotide sequence encoding SEQ ID NO: 11 or a sequence having at least 85%, 86%, 87%, 88%, 89%, 90%, 91%, 92%, 93%, 94%, 95%, 96%, 97%, 98% or 99% sequence identity to SEQ ID NO: 11; and a nucleotide sequence encoding SEQ ID NO: 16 or a sequence having at least 85%, 86%, 87%, 88%, 89%, 90%, 91%, 92%, 93%, 94%, 95%, 96%, 97%, 98% or 99% sequence identity to SEQ ID NO: 16.

In certain embodiments, the invention relates to a polynucleotide comprising a nucleotide sequence encoding SEQ ID NO: 12 or a sequence having at least 85%, 86%, 87%, 88%, 89%, 90%, 91%, 92%, 93%, 94%, 95%, 96%, 97%, 98% or 99% sequence identity to SEQ ID NO: 12; and a nucleotide sequence encoding SEQ ID NO: 17 or a sequence having at least 85%, 86%, 87%, 88%, 89%, 90%, 91%, 92%, 93%, 94%, 95%, 96%, 97%, 98% or 99% sequence identity to SEQ ID NO: 17.

In certain embodiments, the invention relates to a polynucleotide comprising a nucleotide sequence encoding SEQ ID NO: 13 or a sequence having at least 85%, 86%, 87%, 88%, 89%, 90%, 91%, 92%, 93%, 94%, 95%, 96%, 97%, 98% or 99% sequence identity to SEQ ID NO: 13; and a nucleotide sequence encoding SEQ ID NO: 18 or a sequence having at least 85%, 86%, 87%, 88%, 89%, 90%, 91%, 92%, 93%, 94%, 95%, 96%, 97%, 98% or 99% sequence identity to SEQ ID NO: 18.

In certain embodiments, the invention relates to a polynucleotide comprising a nucleotide sequence encoding SEQ ID NO: 14 or a sequence having at least 85%, 86%, 87%, 88%, 89%, 90%, 91%, 92%, 93%, 94%, 95%, 96%, 97%, 98% or 99% sequence identity to SEQ ID NO: 14; and a nucleotide sequence encoding SEQ ID NO: 19 or a sequence having at least 85%, 86%, 87%, 88%, 89%, 90%, 91%, 92%, 93%, 94%, 95%, 96%, 97%, 98% or 99% sequence identity to SEQ ID NO: 19.

In certain embodiments, the invention relates to a polynucleotide comprising a nucleotide sequence encoding SEQ ID NO: 15 or a sequence having at least 85%, 86%, 87%, 88%, 89%, 90%, 91%, 92%, 93%, 94%, 95%, 96%, 97%, 98% or 99% sequence identity to SEQ ID NO: 15; and a nucleotide sequence encoding SEQ ID NO: 20 or a sequence having at least 85%, 86%, 87%, 88%, 89%, 90%, 91%, 92%, 93%, 94%, 95%, 96%, 97%, 98% or 99% sequence identity to SEQ ID NO: 20.

In certain embodiments, the invention relates to a polynucleotide comprising a nucleotide sequence as defined by SEQ ID NO: 21 or a sequence having at least 85%, 86%, 87%, 88%, 89%, 90%, 91%, 92%, 93%, 94%, 95%, 96%, 97%, 98% or 99% sequence identity to SEQ ID NO: 21; and a nucleotide sequence as defined by SEQ ID NO: 26 or a sequence having at least 85%, 86%, 87%, 88%, 89%, 90%, 91%, 92%, 93%, 94%, 95%, 96%, 97%, 98% or 99% sequence identity to SEQ ID NO: 26.

In certain embodiments, the invention relates to a polynucleotide comprising a nucleotide sequence as defined by SEQ ID NO: 22 or a sequence having at least 85%, 86%, 87%, 88%, 89%, 90%, 91%, 92%, 93%, 94%, 95%, 96%, 97%, 98% or 99% sequence identity to SEQ ID NO: 22; and a nucleotide sequence as defined by SEQ ID NO: 27 or a sequence having at least 85%, 86%, 87%, 88%, 89%, 90%, 91%, 92%, 93%, 94%, 95%, 96%, 97%, 98% or 99% sequence identity to SEQ ID NO: 27.

In certain embodiments, the invention relates to a polynucleotide comprising a nucleotide sequence as defined by SEQ ID NO: 23 or a sequence having at least 85%, 86%, 87%, 88%, 89%, 90%, 91%, 92%, 93%, 94%, 95%, 96%, 97%, 98% or 99% sequence identity to SEQ ID NO: 23; and a nucleotide sequence as defined by SEQ ID NO: 28 or a sequence having at least 85%, 86%, 87%, 88%, 89%, 90%, 91%, 92%, 93%, 94%, 95%, 96%, 97%, 98% or 99% sequence identity to SEQ ID NO: 28.

In certain embodiments, the invention relates to a polynucleotide comprising a nucleotide sequence as defined by SEQ ID NO: 24 or a sequence having at least 85%, 86%, 87%, 88%, 89%, 90%, 91%, 92%, 93%, 94%, 95%, 96%, 97%, 98% or 99% sequence identity to SEQ ID NO: 24; and a nucleotide sequence as defined by SEQ ID NO: 29 or a sequence having at least 85%, 86%, 87%, 88%, 89%, 90%, 91%, 92%, 93%, 94%, 95%, 96%, 97%, 98% or 99% sequence identity to SEQ ID NO: 29.

In certain embodiments, the invention relates to a polynucleotide comprising a nucleotide sequence as defined by SEQ ID NO: 25 or a sequence having at least 85%, 86%, 87%, 88%, 89%, 90%, 91%, 92%, 93%, 94%, 95%, 96%, 97%, 98% or 99% sequence identity to SEQ ID NO: 25; and a nucleotide sequence as defined by SEQ ID NO: 30 or a sequence having at least 85%, 86%, 87%, 88%, 89%, 90%, 91%, 92%, 93%, 94%, 95%, 96%, 97%, 98% or 99% sequence identity to SEQ ID NO: 30.

In some embodiments, the polynucleotide of the invention is transfected with the support of a transfection enhancer, e.g., a transfection enhancer selected from the group of oligonucleotides, lipoplexes, polymersomes, polyplexes, dendrimers, inorganic nanoparticles and cell-penetrating peptides.

In some embodiments, the polynucleotide of the invention is operably linked with another nucleic acid sequence. For instance, a transcription regulatory sequence is operably linked to the polynucleotide of the invention.

In certain embodiments, the invention relates to a host cell comprising the polynucleotide of the invention.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell but may contain mutations. Mutant progeny that have the same function or biological activity as screened or selected for in the originally transformed cell are included herein.

In certain embodiments the host cell is directly or indirectly used in therapy (e.g., cell therapy).

In certain embodiments a method for cell therapy comprises the steps of (i) obtaining a cell from a subject; (ii) transform the cell using a tool (e.g. a vector) comprising the polynucleotide of the invention and/or transform the cell to produce the antibody of the invention; and (iii) administering the transformed cell to a subject.

In certain embodiments, the subject in step (i) and step (iii) of the method for cell therapy are the same subject.

In certain embodiments, the subject in step (i) and step (iii) of the method for cell therapy are different subjects.

In certain embodiments, the subject in step (i) and step (iii) of the method for cell therapy are different subjects that belong to different species.

In certain embodiments, the subject in step (i) of the method for cell therapy is a subject from the genus Sus and the subject in step (iii) of the method for cell therapy is a subject from the species Homo sapiens.

In certain embodiments, the host cell is a stem cell. In other embodiments, the host cell is a differentiated cell.

In certain embodiments, the host cell is a cell selected from the group of human embryonic stem cells, induced pluripotent stem cells, neural stem cells, mesenchymal stem cells, hematopoietic stem cells and cardiac stem cells.

In certain embodiments, the invention relates to a method for producing an antibody comprising culturing the host cell of the invention.

In a particular embodiment, the method of producing an antibody comprises culturing the host cell of the invention under conditions suitable to allow efficient production of the antibody of the invention.

This production is based, for example, on the immunization of animals, like mice. However, also other animals for the production of antibody/antisera are envisaged within the present invention. For example, monoclonal and polyclonal antibodies can be produced by rabbit, mice, goats, donkeys and the like. The polynucleotide encoding a correspondingly chosen polypeptide of Gremlin-1 can be subcloned into an appropriated vector, wherein the recombinant polypeptide is to be expressed in an organism being able for an expression, for example in bacteria. Thus, the expressed recombinant protein can be intra-peritoneally injected into a mouse and the resulting specific antibody can be, for example, obtained from the mice serum being provided by intra-cardiac blood puncture. The present invention also envisages the production of specific antibodies against native polypeptides and recombinant polypeptides by using a DNA vaccine strategy as exemplified in the appended examples. DNA vaccine strategies are well-known in the art and encompass liposome-mediated delivery, by gene gun or jet injection and intramuscular or intradermal injection. Thus, antibodies directed against a polypeptide or a protein or an epitope of Gremlin-1, in particular the epitope of the antibodies provided herein, can be obtained by directly immunizing the animal by directly injecting intramuscularly the vector expressing the desired polypeptide or a protein or an epitope of Gremlin-1, in particular the epitope of the antibodies of the invention. The amount of obtained specific antibody can be quantified using an ELISA, which is also described herein below. Further methods for the production of antibodies are well known in the art, see, e.g. Harlow and Lane, 1988, CSH Press, Cold Spring Harbor.

In a particular embodiment, the method of producing an antibody comprises culturing the host cell of the invention under conditions suitable to allow efficient production of the antibody of the invention.

In one such embodiment, a host cell comprises (e.g., has been transformed with): (1) a vector comprising a nucleic acid that encodes an amino acid sequence comprising the VL of the antibody and an amino acid sequence comprising the VH of the antibody of the invention, or (2) a first vector comprising a nucleic acid that encodes an amino acid sequence comprising the VL of the antibody and a second vector comprising a nucleic acid that encodes an amino acid sequence comprising the VH of the antibody of the invention. In one embodiment, the host cell is eukaryotic, e.g. a Chinese Hamster Ovary (CHO) cell or lymphoid cell (e.g., YO, NSO, Sp20). In one embodiment, a method of making an antibody specifically binding to Gremlin-1 is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody specifically binding to Gremlin-1, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523; Charlton, 2003, Methods in Molecular Biology, Vol. 248; BKC Lo, 2003, Humana Press, pp. 245-254. After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, 2004, Nat. Biotech. 22:1409-1414, and Li et al., 2006, Nat. Biotech. 24:210-215.

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177; 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are macaque kidney CVI line transformed by SV40 (COS-7); human embryonic kidney line (293 or 293 cells as described, e.g., in Graham et al., 1997, J. Gen Viral. 36:59); baby hamster kidney cells (BHK); mouse sertoli cells (TM4 cells as described, e.g., in Mather, 1980, Biol. Reprod. 23:243-251); macaque kidney cells (CV I); African green macaque kidney cells (VERO-76); human cervical carcinoma cells (HELA); canine kidney cells (MDCK; buffalo rat liver cells (BRL 3A); human lung cells (WI38); human liver cells (Hep G2); mouse mammary tumor (MMT 060562); TRI cells, as described, e.g., in Mather et al., 1982, Annals N. Y Aead. Sei. 383:44-68; MRC 5 cells; and FS4 cells. Other useful mammalian host cell lines include Chinese hamster ovary (CHO) cells, including DHFR CHO cells (Urlaub et al., 1980, Proc. Natl. Acad. Sc. USA 77:4216); and myeloma cell lines such as YO, NSO and Sp2/0. For a review of certain mammalian host cell lines suitable for antibody production, see, e.g., Yazaki and Wu, Methods in Molecular Biology, Vol. 248 BKC Lo, 2003., Humana Press, pp. 255-268.

In an embodiment, an antibody specifically binding to Gremlin-1 provided herein may be identified, screened for, or characterized for their physical/chemical properties and/or biological activities by various assays known in the art.

In certain embodiments, the invention relates to a pharmaceutical composition comprising the antibody, or antigen-binding fragment thereof, according to the invention, the polynucleotide of the invention or the host cell of the invention, and a pharmaceutically acceptable carrier.

The term “pharmaceutical composition”, as used herein, refers to a preparation which is in such form as to permit the biological activity of an active ingredient contained therein to be effective, and which contains no additional components which are unacceptably toxic to a subject to which the formulation would be administered.

The term “pharmaceutically acceptable carrier”, as used herein, refers to an ingredient in a pharmaceutical formulation, other than an active ingredient, which is nontoxic to a subject. A pharmaceutically acceptable carrier includes, but is not limited to, a buffer, excipient, stabilizer, or preservative.

Pharmaceutical compositions of the antibody, or antigen-binding fragment thereof, the polynucleotide, the host cell as described herein are prepared by mixing such antibody/antigen-binding fragment/polynucleotide/host cell having the desired degree of purity with one or more optional pharmaceutically acceptable carriers (Osol et al., 1980 Remington's Pharmaceutical Sciences 16th edition), in certain examples, in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US 2005/0260186 and US 2006/0104968. In one aspect, a sHASEGP is combined with one or more additional glycosaminoglycanases such as chondroitinases.

Exemplary lyophilized antibody compositions are described in U.S. Pat. No. 6,267,958. Aqueous antibody compositions include those described in U.S. Pat. No. 6,171,586 and WO 2006/044908, the latter formulations including a histidine-acetate buffer.

Active ingredients of the pharmaceutical composition may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions. Such techniques are disclosed in Osol et al., 1980, Remington's Pharmaceutical Sciences 16th edition.

Sustained-release preparations may be prepared. Suitable examples of sustained-release preparations include semipermeable matrices of solid hydrophobic polymers containing the antibody, or antigen-binding fragment thereof, of the invention and/or the polynucleotide of the invention, which matrices are in the form of shaped articles, e.g. films, or microcapsules. The pharmaceutical compositions to be used for in vivo administration are generally sterile. Sterility may be readily accomplished, e.g., by filtration through sterile filtration membranes.

In some embodiments comprising the polynucleotide of the invention, the pharmaceutically acceptable carrier is or enables formation of a retrovirus, an adenovirus, an adeno-associated virus, an envelope protein pseudotyping a viral vector, a replication-competent vector, cis and trans-acting elements, a herpes simplex virus and/or parts thereof.

In some embodiments comprising the host cell of the invention, the pharmaceutically acceptable carrier enables conservation and/or viability of cells.

The composition herein may also contain more than one active ingredient as necessary for the particular indication being treated, preferably those with complementary activities that do not adversely affect each other.

In certain embodiments, the invention relates to the pharmaceutical composition according to the invention, comprising at least one further therapeutic agent.

The term “therapeutic agent”, as used herein, refers to a compound or a composition of matter that upon administration to a subject in a therapeutically effective amount, provides a therapeutic benefit to the subject. A therapeutic agent may be any type of drug, medicine, pharmaceutical, hormone, antibiotic, protein, gene, growth factor, bioactive material, used for treating, controlling, or preventing diseases or medical conditions. Those skilled in the art will appreciate that the term “therapeutic agent” is not limited to drugs that have received regulatory approval. In some embodiments, one or more therapeutic agents is selected from the group of anti-inflammatory agent, immunomodulator, antigenic peptide, antibiotic, diuretic, loop diuretic, potassium sparing agent, vasodilator, ACE inhibitor, angiotensin II antagonist, positive inotropic agent, phosphodiesterase inhibitor, beta-adrenergic receptor antagonist, calcium channel blocker, nitrate, alpha blocker, central alpha antagonist, statin, and a combination of these agents.

In some embodiments, the further therapeutic agent may be useful to reduce the possible side-effect(s) associated with the administration of an antibody, or an antigen-binding fragment thereof, of the invention.

In some embodiments, the further therapeutic agent may be useful to support the effect associated with the administration of an antibody, or an antigen-binding fragment thereof, of the invention.

In some embodiments, administration of the further therapeutic and an antibody, or an antigen-binding fragment thereof, of the invention results in a synergistic effect regarding desired effect and/or side effect.

The further therapeutic agent may be selected from the group of a small molecule drug, a protein/polypeptide, an antibody, molecule drug with antibiotic activity, phage-based therapy, a nucleic acid molecule or a siRNA in a form of natural or synthetic derivatives.

Such combination therapies noted above encompass combined administration (where two or more therapeutic agents are included in the same or separate formulations), and separate administration, in which case, administration of the antibody, or antigen-binding fragment thereof, of the invention can occur prior to, simultaneously, and/or following, administration of the additional therapeutic agent and/or carrier.

In some embodiments, the pharmaceutical composition of the invention (and any additional therapeutic agent) is administered systemically. In some embodiments, the pharmaceutical composition of the invention (and any additional therapeutic agent) is administered locally. In some embodiments, the pharmaceutical composition of the invention (and any additional therapeutic agent) is administered by any suitable means, including parenteral, intrapulmonary, and intranasal, and, if desired for local treatment, intralesional, intrauterine or intravesical administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

In some cases, an effective amount of the pharmaceutical composition of the invention (and any additional therapeutic agent) can be any amount that reduces the severity, or occurrence, of symptoms of the disease, disorder and/or condition to be treated without producing significant toxicity to the subject. In some cases, an effective amount of the pharmaceutical composition of the invention (and any additional therapeutic agent) can be any amount that reduces the number of diseased cells (e.g., dysregulated immune cells), pathogens and/or infected cells without producing significant toxicity to the subject.

The effective amount of the pharmaceutical composition of the invention (and any additional therapeutic agent) can remain constant or can be adjusted as a sliding scale or variable dose depending on the subject's response to treatment. In some cases, the frequency of administration can be any frequency that reduces the severity, or occurrence, of symptoms of the disease, disorder and/or condition to be treated without producing significant toxicity to the subject. Various factors can influence the actual effective amount used for a particular application. For example, the frequency of administration, duration of treatment, use of multiple treatment agents, route of administration, and severity of the disease, disorder and/or condition may require an increase or decrease in the actual effective amount administered.

In some cases, the frequency of administration can be any frequency that reduces the number of diseased cells (e.g., dysregulated immune cells), pathogens and/or infected cells without producing significant toxicity to the subject. As with the effective amount, various factors can influence the actual frequency of administration used for a particular application. For example, the effective amount, duration of treatment, use of multiple treatment agents, route of administration, and severity of the disease, disorder and/or condition may require an increase or decrease in administration frequency.

In some cases, an effective duration for administering the pharmaceutical composition of the invention (and any additional therapeutic agent) can be any duration that reduces the severity, or occurrence, of symptoms of the disease, disorder and/or condition to be treated without producing significant toxicity to the subject. In some cases, an effective duration for administering the pharmaceutical composition of the invention (and any additional therapeutic agent) can be any duration that reduces the number of diseased cells (e.g., dysregulated immune cells), pathogens and/or infected cells without producing significant toxicity to the subject. Multiple factors can influence the actual effective duration used for a particular treatment. For example, an effective duration can vary with the frequency of administration, effective amount, use of multiple treatment agents, route of administration, and severity of the disease, disorder and/or condition being treated.

In certain embodiments, a course of treatment and/or the severity of the disease, disorder and/or condition being treated can be monitored. Any appropriate method can be used to determine whether or not the severity of a disease, disorder and/or condition is reduced. For example, the severity of a disease (e.g., inflammation) can be assessed in some embodiments using imaging techniques (with or without contrast), biopsy techniques, colonoscopy, sigmoidoscopy, digital rectal exam, blood assay, platelet assay, fecal assay, urine assay, endoscopic techniques, ELISA techniques, PCR-based techniques, blotting techniques (e.g., western blot), flow cytometry, genetic analysis (e.g., for gene rearrangements), and/or histological techniques at different time points. For example, the severity of an infection can be assessed using antibody techniques, viral antigen detection tests, culturing techniques, ELISA techniques, PCR-based techniques (e.g., viral load test), blotting techniques (e.g., western blot), and/or histological techniques at different time points.

Any appropriate method can be used to monitor the response to therapies with the pharmaceutical composition of the invention and/or the antibody, or antigen binding fragment thereof, of the invention. For example, techniques to detect levels ingredients of the pharmaceutical composition (e.g. the antibody, or antigen-binding fragment thereof, of the invention, the polynucleotide of the invention, the host cell of the invention and/or a further therapeutic agent) including ELISA techniques, PCR-based techniques, blotting techniques (e.g., western blot), hybridization techniques (e.g., ISH) and/or histological techniques (e.g., IHC).

The pharmaceutical composition of the invention (and any additional therapeutic agent) would be formulated, dosed, and administered in a fashion consistent with good medical practice. Factors for consideration in this context include the particular disorder being treated, the particular subject being treated, the clinical condition of the individual patient, the cause of the disorder, the site of delivery of the agent, the method of administration, the scheduling of administration, and other factors known to medical practitioners. An antibody, or an antigen-binding fragment thereof, of the invention need not be, but is optionally formulated with one or more further therapeutic agents currently used to prevent or treat the disorder in question. The effective amount of such other agents depends on the amount of antibody, or antigen-binding fragment thereof, present in the composition, the type of disorder or treatment, and other factors for consideration discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody, or an antigen-binding fragment thereof, of the invention (when used alone or in combination with one or more other further therapeutic agents) will depend on the type of disease to be treated, the type of antibody, or antigen-binding fragment thereof, the severity and course of the disease, whether the antibody or antigen-binding fragment is administered for preventive or therapeutic purposes, previous therapy, the patient's clinical history and response to the antibody or antigen-binding fragment and the discretion of the attending physician.

The antibody, or antigen-binding fragment thereof, of the invention and/or the antibody used as a further therapeutic agent are/is suitably administered to the patient at one time or over a series of treatments. Depending on the type and severity of the disease, about 1 μg/kg to 15 mg/kg (e.g. 0.1 mg/kg-10 mg/kg) of antibody or antigen-binding fragment can be an initial candidate dosage for administration to the patient, whether, for example, by one or more separate administrations, or by continuous infusion. One typical daily dosage might range from about 1 μg/kg to 100 mg/kg or more, depending on the factors for consideration mentioned above. For repeated administrations over several days or longer, depending on the condition, the treatment would generally be sustained until a desired suppression of disease symptoms occurs. One exemplary dosage of the antibody or antigen-binding fragment would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose followed by one or more lower doses may be administered. However, other dosage regimens may be useful. The progress of this therapy is easily monitored by conventional techniques and assays.

In some embodiments wherein the further therapeutic agent is an antibody, the antibodies are linked, e.g., covalently linked. In some embodiments the antibody, or fragment thereof, of the invention and the further therapeutic agent embody a fusion antibody.

In some embodiments wherein the further therapeutic agent is a small molecule, one dose of the pharmaceutical composition comprises about 1 μg/kg to 15 mg/kg (e.g., 0.1 mg/kg-10 mg/kg) of the small molecule, depending on the factors for consideration mentioned above.

In some embodiments wherein the further therapeutic agent is a small molecule, the small molecule is linked (e.g., covalently linked) to the antibody, or antigen-binding fragment thereof, of the invention.

In other embodiments, the further therapeutic agent is in a different modified-release formulation than the antibody, or antigen-binding fragment thereof, of the invention. For example, the further therapeutic agent but not the antibody, or antigen-binding fragment thereof, is bound to a release extender or vice versa. This can be useful to adjust for pharmacokinetic and/or pharmacodynamic differences between the further therapeutic agent and the antibody, or antigen-binding fragment thereof.

In other embodiments the further therapeutic agent enables target delivery of the antibody, or antigen-binding fragment thereof, of the invention. For example, the antibody, or antigen-binding fragment thereof, of the invention achieves a higher concentration in cardiac tissue compared to other tissues via delivery mediated by the further therapeutic agent.

In some embodiments the antibody, or antigen-binding fragment thereof, of the invention enables target delivery of the further therapeutic agent. For example, the further therapeutic agent achieves a higher concentration in a Gremlin-1 associated cell, a Gremlin-1 associated cell environment, a Gremlin-1 associated tissue and/or a Gremlin-1 associated organ compared to other sites via delivery mediated by the antibody, or antigen-binding fragment thereof, of the invention.

Methods for targeted delivery mediated by active agents are well known in the art (see, e.g., US 2006/0142202; U.S. Pat. No. 8,491,914).

In some embodiments pharmaceutical composition comprises the polynucleotide of the invention and the further therapeutic agent is a transfection enhancer, e.g., a transfection enhancer selected from the group of oligonucleotides, lipoplexes, polymersomes, polyplexes, dendrimers, inorganic nanoparticles and cell-penetrating peptides.

In some embodiments the pharmaceutical composition comprises the polynucleotide in the form of a vector genome in doses in the range from at least 10⁶, 10⁷, 10⁸, 10⁹, 10¹⁰, 10¹¹, 10¹², 10¹³, 10¹⁴, 10¹⁵, 10¹⁶, or more, vector genomes per kilogram (vg/kg) of the weight of the subject, to achieve a therapeutic effect.

In some embodiments the pharmaceutical composition comprises host cell and the further therapeutic agent is a cell signaling molecule such as a hormone, a neurotransmitter or a cytokine (see, e.g., Ding, Z. et al., 2017 Sci Rep 7, 12168) In certain embodiments the pharmaceutical composition comprises a clinically relevant number or population of host cells and/or stem cell therapy cells, e.g., at least 10⁴, 10⁵, 10⁶, 10⁷, 10⁸, 10⁹, typically more than 10⁹ or at least 10¹⁰ cells per dose. The number of cells will depend upon the ultimate use for which the pharmaceutical composition is intended as will the type of cell. In some embodiments, the pharmaceutical composition will contain greater than 70%, generally greater than 80%, 85% and 90-95% of the host cells and/or stem cell therapy cells. For uses provided herein, the cells are typically in a volume of a liter or less, can be 500 ml or less, even 250 ml or 100 ml or less. Hence the density of the desired cells is typically be greater than 10⁶ cells/ml and generally is greater than 10⁷ cells/ml. The clinically relevant number of host cells can be apportioned into multiple infusions that cumulatively equal or exceed 10⁹, 10¹⁰ or 10¹¹ cells.

The total dose of the host cell of the invention for one therapy cycle is typically about 1×10⁴ cells/kg to 1×10¹⁰ cells/kg host cells or more, depending on the factors for consideration mentioned above.

In some embodiments any pharmaceutical composition is used for any of the methods or used in the treatments described herein.

In certain embodiments, the invention relates to the pharmaceutical composition according to the invention, wherein the further therapeutic agent is selected from the group consisting of an anti-inflammatory agent, an immunomodulator, an antibiotic, an angiotensin-converting-enzyme inhibitor, a β-blocker and a diuretic.

The term “anti-inflammatory agent”, as used herein, refers therapeutic agents for the treatment of an inflammatory disease or the symptoms associated therewith. Anti-inflammatory agents include, without limitation, non-steroidal anti-inflammatory drugs (NSAIDs; e.g., aspirin, ibuprofen, naproxen, methyl salicylate, diflunisal, indomethacin, sulindac, diclofenac, ketoprofen, ketorolac, carprofen, fenoprofen, mefenamic acid, piroxicam, meloxicam, methotrexate, celecoxib, valdecoxib, parecoxib, etoricoxib, and nimesulide), corticosteroids (e.g., prednisone, betamethasone, budesonide, cortisone, dexamethasone, hydrocortisone, methylprednisolone, prednisolone, tramcinolone, and fluticasone), rapamycin (see, e.g., Migita et al. 1997, Clin. Exp. Immunol. 108:199-203; Migita et al., 1996, Clin. Exp. Immunol. 104:86-91; Foroncewicz et al., 2005, Transpl. Int. 18:366-368), high density lipoproteins (HDL) and HDL-cholesterol elevating compounds (see, e.g., Birjmohun et al., 2007, Arterioscler. Thromb. Vase. Biol., 27:1153-1158; Nieland et al., 2007, J. Lipid Res., 48:1832-1845; Bloedon et al., 2008, J. Lipid Res., Samaha et al., 2006, Arterioscler. Thromb. Vase. Biol., 26:1413-1414, which discloses the use of rosiglitazone as an anti-inflammatory, Duffy et al., 2005 Curr. Opin. Cardiol., 20:301-306), rho-kinase inhibitors (see, e.g., Hu, E., 2006, Rec. Patents Cardiovasc. Drug Discov., 1:249-263), anti-malarial agents (e.g., hydroxychloroquine and chloroquine), acetaminophen, glucocorticoids, steroids, beta-agonists, anticholinergic agents, xanthine derivatives (e.g., methyl xanthines), gold injections (e.g., sodium aurothiomalate), sulphasalazine, penicillamine, anti-angiogenic agents, dapsone, psoralens, anti-viral agents, anti TNF agents, anti-IL-1 agents and statins (see, e.g., Paraskevas et al., 2007, Curr. Pharm. Des., 13:3622-36; Paraskevas, K. I., 2008, Clin. Rheumatol. 27:281-287), preferably infliximab, adalimumab, certolizumab pegol, golimumab, etanercept, curcumin, IL-1RA, rilonacept, canakinumab, allopurinol, colchicine, prednisone, pentoxifylline, rosuvastatin, oxypurinol.

In some embodiments, the anti-inflammatory effect of the anti-inflammatory agent and the anti-inflammatory effect of the antibody, or antigen-binding fragment thereof, of the invention, are additive, preferably synergistic.

In some embodiments, the anti-inflammatory effect of the anti-inflammatory agent reduces the immune reaction against the antibody, or antigen-binding fragment thereof, of the invention.

The term “immunomodulator”, as used herein, refers to a therapeutic agent which modulates the immune system of a subject. The immune modulator may adjust the immune response to a desired level, as in immunopotentiation, immunosuppression, or induction of immunologic tolerance. Immune modulators for use in compounds of the invention include, but are not limited to, proteins, peptides, antibodies, antibody fragments, small molecules, cytokines, hormones, enzymes, nucleic acids, antisense oligonucleotides such as siRNA, toxins, anti-angiogenic agents, cytotoxic agents, pro-apoptotic agents, stem cell-based therapy and other known therapeutic agents. Preferred immune modulators include antigenic peptides, small molecules (for example, R848, Loxoribine, Stat-3 inhibitors, TGF-β inhibitors, Rapamycin/FK506), cytokines (for example, IL-2, TGF-β), antibody fragments (for example, CTLA-4 agonist scFv) and/or nucleic acids (for example, CpG, siRNA), preferably antigenic peptides, immunoglobulin, methotrexate and/or stem cell-based therapy. The immune modulator may modulate cytokine and/or chemokine biosynthesis.

In some embodiments, the immunomodulatory effect of the immunomodulator and the immunomodulatory effect of the antibody, or antigen-binding fragment thereof, of the invention, are additive, preferably synergistic.

In some embodiments, the immunomodulatory effect of the immunomodulator reverses undesired effect of the antibody, or antigen-binding fragment thereof, of the invention on the immune system.

In some embodiments, the immunomodulatory effect of the immunomodulator reduces the immune reaction against the antibody, or antigen-binding fragment thereof, of the invention.

The term “antibiotic”, as used herein, refers to as used herein, refers to a therapeutic agent with properties useful in the treatment against a bacteria-related disease. An antibiotic may have, inter alia, properties of preventing, inhibiting, suppressing, reducing, adversely impacting, and/or interfering with the growth, survival, replication, function, and/or dissemination of a bacterium. In some embodiments, the antibiotic comprises or consists of anti-bacterial phages. Classes of antibiotics include, but are not limited to, macrolides (e.g., erythromycin), penicillins (e.g., nafcillin), cephalosporins (e.g., cefazolin), carbapenems (e.g., imipenem), monobactam (e.g., aztreonam), other beta-lactam antibiotics, beta-lactam inhibitors (e.g., sulbactam), oxalines (e.g. linezolid), aminoglycosides (e.g., gentamicin), chloramphenicol, sufonamides (e.g., sulfamethoxazole), glycopeptides (e.g., vancomycin), quinolones (e.g., ciprofloxacin), tetracyclines (e.g., minocycline), fusidic acid, trimethoprim, metronidazole, clindamycin, mupirocin, rifamycins (e.g., rifampin), streptogramins (e.g., quinupristin and dalfopristin) lipoprotein (e.g., daptomycin) and polyenes (e.g., amphotericin B). Examples of specific antibiotics include, but are not limited to, erythromycin, nafcillin, cefazolin, imipenem, aztreonam, gentamicin, sulfamethoxazole, vancomycin, ciprofloxacin, trimethoprim, rifampin, metronidazole, clindamycin, teicoplanin, mupirocin, azithromycin, clarithromycin, ofloxacin, lomefloxacin, norfloxacin, nalidixic acid, sparfloxacin, pefloxacin, amifloxacin, gatifloxacin, moxifloxacin, gemifloxacin, enoxacin, fleroxacin, minocycline, linezolid, temafloxacin, tosufloxacin, clinafloxacin, sulbactam, clavulanic acid and any combination thereof, preferably rifaximin, vancomycin, or trimethoprim-sulfamethoxazole.

In some embodiments, the effect of the antibiotic and the effect of the antibody, or antigen-binding fragment thereof, of the invention, are complement each other, preferably synergistic. For example, the antibiotics supports the reduction of the number of bacteria in a certain subject and the antibody, or antigen-binding fragment thereof, of the invention, reduces the effects of the infection. In some embodiments, the antibiotic additionally has anti-inflammatory properties.

The term “angiotensin-converting-enzyme inhibitor”, as used herein, refers to any therapeutic agent inhibiting the activity of Angiotensin-Converting-Enzyme or any ACE like activity leading to a reduced formation of Ang 1-8 from Ang 1-10, or a pro-drug thereof. ACE is an enzyme involved in the RAS, in particular in the degradation and formation of angiotensins. Similar to chymase, ACE is a carboxypeptidase and converts Ang I to Ang 1-8. ACE is a metalloprotease that is built up by an N-terminal and a C-terminal domain. The two domains possess different substrate specificities and their slightly different molecular structure can also result in differences in the affinity of ACE inhibitors for the two individual domains. Moreover, different isoforms of ACE are known to be expressed in humans, including membrane attached, soluble, full length and truncated forms. In an embodiment, ACE inhibitors include inhibitors of ACE and ACE isoforms. ACE is broadly expressed throughout multiple tissues and fluids of the human body (Maluf-Meiken, Leila C V et al., 2012, International journal of hypertension 2012:581780; Hattori, Monica A., et al., 2000 Hypertension 35.6: 1284-1290, Deddish, Peter A., et al., 1998, Hypertension 31.4: 912-917). Accordingly, in an embodiment, the ACE inhibitor inhibits the conversion of Ang I to Ang 1-8. In an embodiment, the conversion of Ang I to Ang 1-8 is inhibited by the ACE inhibitor by at least 10, 20, 30, 40, 50, 60, 70, 80, or 90%, or any range in between these devalues. In one embodiment, the ACE inhibitor is a small molecule. In an alternative embodiment, the ACE inhibitor is a protein or peptide, e.g. an antibody, or an inhibitory nucleic acid, such as a siRNa, shRNA, miRNA, or a vector encoding such nucleic acids. The ACE inhibitor may be selected from the group consisting of alacepril, benazepril, benazeprilat, captopril, ceronapril, cilazapril, delapril, enalapril, enalaprilat, fosinopril, imidapril, lisinopril, moexipril, moveltopril, perindopril, quinapril, quinaprilat, ramipril, ramiprilat, spirapril, temocapril, trandolapril, zofenopril, and pharmaceutically acceptable salts thereof. In one embodiment, the ACE inhibitor is selected from agents that have been marketed already, e.g. benazepril, benazaprilat, ramipril and ramiprilat, quinapril, quinaprilat, lisinopril, trandolapril, enalapril, or enalaprilat.

The term “beta-blocker”, as used herein, refers to a therapeutic agent to block the β 1-, β 2-, or β 3-adrenergic receptor in sympathetic nerves, including, but not limited to, landiolol, esmolol, propranolol, metoprolol, bisoprolol, acebutolol, atenolol, bufetolol, arotinolol, carteolol, pindolol, alprenolol, sotalol, nadolol, bopindolol, timolol, indenolol, bunitrolol, penbutolol, nipradilol, tilisolol, celiprolol, betaxolol, practolol, carvedilol, amosulalol, labetalol, bevantolol, oxprenolol, and levobunolol, as well as a salt thereof. Short-acting beta-blockers include, for example, landiolol, esmolol or a salt thereof. Intravenous beta-blockers include, for example, landiolol, esmolol, propranolol, labetalol, sotalol, metoprolol, or a salt thereof.

The term “diuretic”, as used herein, refers to any drug that elevates the rate of urination and thus provides a means of forced diuresis. There are several categories of diuretics. All diuretics increase the excretion of water from bodies, although each class does so in a distinct way. Diuretics include, without limitation, bendroflumethiazide, chlorthalidone, hydrochlorothiazide, hydroflumethiazide, indapamide, methyclothiazide, metolazone, polythiazide, furosemide and triamterene.

In some embodiments, the effect of the further therapeutic agent and the effect of the antibody, or antigen-binding fragment thereof, of the invention, are complement each other, preferably synergistically complement each other. For example, the therapeutic agent supports the reduction of one aspect of a disease, disorder and/or condition in a subject and the antibody, or antigen-binding fragment thereof, of the invention, reduces another aspect of a disease, disorder and/or condition in a subject. In some embodiments, the effect of the therapeutic agent and the effect of the antibody, or antigen-binding fragment thereof, of the invention are at least partially overlapping.

In certain embodiments, the antibody, or antigen-binding fragment thereof, the polynucleotide, the host cell as described herein is used in a combination therapy noted above encompass combined administration (where two or more therapeutic agents are included in the same or separate pharmaceutical compositions), and separate administration, in which case, administration of the antibody, or antigen-binding fragment thereof, the polynucleotide, the host cell as described herein can occur prior to, simultaneously, and/or following, administration of the additional therapeutic agent and/or adjuvant. The antibody, or antigen-binding fragment thereof, the polynucleotide, the host cell as described herein can also be used in combination with other forms of therapy, e.g., surgery.

In certain embodiments, the invention relates to the pharmaceutical composition according to the invention, wherein the further therapeutic agent is
(i) an anti-inflammatory agent selected from the group consisting of infliximab, adalimumab, certolizumab pegol, golimumab, etanercept, curcumin, IL-1RA, rilonacept, canakinumab, allopurinol, colchicine, prednisone, pentoxifylline, rosuvastatin and oxypurinol;
(ii) an immunomodulator selected from the group consisting of antigenic peptide, immunoglobulin, methotrexate and stem cell-based therapy; or
(iii) an antibiotic selected from the group consisting of anti-bacterial phages, rifaximin, vancomycin, and trimethoprim-sulfamethoxazole.

In certain embodiments, the invention relates to the antibody, or antigen-binding fragment thereof, of the invention, the polynucleotide of the invention, the host cell of the invention, or the pharmaceutical composition of the invention for use as a medicament.

In certain embodiments, the invention relates to the antibody, or antigen-binding fragment thereof, of the invention, the polynucleotide of the invention, the host cell of the invention, or the pharmaceutical composition of the invention for use in treating and/or preventing heart failure and/or an inflammatory disease.

The term “treatment” (and grammatical variations thereof such as “treat” or “treating”), as used herein, refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis. In some embodiments, antibodies of the invention are used to delay development of a disease or to slow the progression of a disease.

The term “prevention”, as used herein, relates to the capacity to prevent, minimize or hinder the onset or development of a disorder, disease or condition before its onset.

The term “heart failure”, as used herein, refers to a cardiac condition that occurs when a problem with the structure and/or function of the heart impairs its ability to supply sufficient blood flow to meet the body's needs. It can cause a variety of symptoms (e.g., chiefly shortness of breath and ankle swelling) but some patients can be symptom free. Heart failure includes, inter alia, left ventricular heart failure (heart failure with reduced ejection fraction (HFrEF)), and heart failure with preserved ejection fraction (HFpEF). Parameters used in diagnosis of heart failure include, without limitation, C-reactive protein (CRP) levels, NT-pro B type natriuretic peptide (NT-ProBNP), creatine kinase (CK), exercise tolerance, left ventricle ejection fraction. In some embodiments the heart failure occurs after myocarditis and/or inflammatory cardiomyopathy. In some embodiments heart failure is caused by myocarditis and/or inflammatory cardiomyopathy.

The term “inflammatory disease”, as used herein, refers to a disease, a disorder and/or a condition that is characterized by increased inflammation. Inflammation is characterized by a dysregulation of inflammation markers and/or increased immune cell infiltration, activation, proliferation, and/or differentiation in the blood, in a tissue, in an organ and/or in a certain cell-type. In some embodiments, the inflammatory disease is an inflammatory cardiac disease.

Cells involved in inflammation include, without limitation T cells, monocytes, neutrophils, blood vessels, fibroblasts and/or cardiomyocytes. Symptoms of inflammation may be detectable by laboratory tests and/or can manifest clinically.

Clinical symptoms of inflammation include, inter alia, pain, heat, redness, swelling and/or loss of function.

An inflammation marker is a marker that is indicative for inflammation in a subject. Inflammatory markers include, without limitation, CRP, erythrocyte sedimentation rate (ESR), and procalcitonin (PCT), Interleukin (e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-33, IL-32, IL-33, IL-35 or IL-36) Tumor necrosis factor (e.g., TNF alpha, TNF beta), Interferon (e.g., interferon alpha, interferon beta, interferon gamma) MIP-I, MCP-I, RANTES, other chemokines and/or other cytokines. An inflammatory marker may also be detectable indirectly, e.g., by detection of an inhibitory factor of an inflammatory marker (e.g., binding factor and/or antagonist). In some embodiments, the inflammatory marker is measured in cells involved in inflammation, in cells affected by cells involved in inflammation, in a tissue, and/or in the blood. In some embodiments, the inflammation marker is indicative for immune cell infiltration, activation, proliferation and/or differentiation. Detection of the inflammation marker or the ratio of two or more inflammation markers is detected outside the normal range. The normal range of inflammation markers and whether a marker (ratio) has to be below or above a threshold to be indicative for inflammation is known to the person skilled in the art. In some embodiments, the gene expression level, the RNA transcript level, the protein expression level, the protein activity level and/or the enzymatic activity level of at least one inflammation marker is detected. In some embodiments at least one inflammation marker is detected quantitatively and/or qualitatively.

Causes of inflammation include without limitation physical injury, ionizing radiation, infections (e.g., by pathogens), immune reactions due to hypersensitivity, cancer, chemical irritants, medications, toxins, alcohol and nutrients (e.g., nutrient excess).

Inflammatory diseases and heart failure have common underlying mechanisms. For example, CRP is an important marker for both inflammation and heart failure (Schmalgemeier H et al., 2014 Respiration 87:113-120) and recent literature confirms that inflammation and heart failure are linked (see e.g., Sorriento D et al., 2019, International journal of molecular sciences vol. 20, 16 3879). The technical effect provided with the invention is useful for heart failure and inflammatory diseases. A common complication of inflammatory diseases and heart failure is the formation of fibrotic tissue, which can result in reduced function and/or loss of function of a tissue and/or an organ. In certain embodiments, the antibody, or the antigen-binding fragment thereof, of the invention is used for the treatment and/or prevention of an inflammatory diseases and/or heart failure and reduces and/or prevents formation of fibrotic tissue. In certain embodiments, the antibody, or the antigen-binding fragment thereof, of the invention is used for the treatment and/or prevention of fibrotic tissue, preferably in the treatment and/or prevention of fibrotic tissue of the heart.

As described herein, antibodies of the invention reduce markers of inflammation and/or markers of heart failure such as CRP and reduce immune cell infiltration, in particular immune cell infiltration in cardiac tissue in a mouse model for inflammatory diseases and/or heart failure (e.g., Ex. 4).

Accordingly, the antibody, or the antigen-binding fragment thereof, of the invention, is surprisingly useful for treating and/or preventing heart failure and/or an inflammatory disease.

In certain embodiments, the invention relates to an antibody, or antigen-binding fragment thereof, according to the invention, for use in treating and/or preventing heart failure.

Patients with heart failure, which have adverse outcome, have a positive correlation between the amount of Gremlin-1, cardiac fibrosis and the degree of cardiac dysfunction in heart failure patients (Mueller K A et al., 2013, J Card Fail, 19(10):678-84).

In some embodiments, the degree of Gremlin-1 expression in the myocardium is used to determine the stage of a disease, disorder and/or condition that increases the risk of heart failure. In some embodiments, the antibody, or the antigen-binding fragment thereof, of the invention is used in treatment of early-stage, to mid stage of a disease, disorder and/or condition that increases the risk of heart failure, preferably in the early stage of a disease, disorder and/or condition that increases the risk of heart failure. The degree of Gremlin-1 expression in the myocardium may be determined by, for example, in cardiac tissue biopsies using immunohistochemistry staining. A person skilled in the art knows how to determine the grade of positive signal by immunohistochemistry in the heart.

In some embodiments, the antibody, or the antigen-binding fragment thereof, of the invention, is used to improve clinical parameters such as CRP levels, NT-ProBNP, CK, exercise tolerance, left ventricle ejection fraction. In some embodiments, the antibody, or the antigen-binding fragment thereof, of the invention, is used for treatment of a disease, condition and/or disorder characterized by parameters such as CRP levels, NT-ProBNP, CK, exercise tolerance, left ventricle ejection fraction. Methods and devices for measuring these parameters are known to the person skilled in the art.

As described herein (e.g., Ex. 4), antibodies of the invention reduce markers of heart failure such as CRP and reduce immune cell infiltration in cardiac tissue.

Accordingly, the antibody, or the antigen-binding fragment thereof, of the invention, are surprisingly useful for use in treating and/or preventing heart failure, particularly when used early in the disease progression.

In certain embodiments, the invention relates to an antibody, or antigen-binding fragment thereof, according to the invention, for use in treating and/or preventing an inflammatory disease.

In some embodiments, the inflammation is primarily mediated by cells of the innate immune system. In some embodiments, the inflammation is primarily mediated by cells of the adaptive immune system.

In some embodiments, the inflammatory disease is characterized by acute inflammation, that is the duration of inflammation symptoms typically takes from about a few minutes (e.g., 2, 5, 10, 15, 30, 45 minutes) to a few days (e.g., 2, 3, 5, 7, 10 or 14 days). Acute inflammation typically occurs upon a stimulus such as infection or injury.

In some embodiments, the inflammatory disease is characterized by chronic inflammation, that is the duration of symptoms of inflammation typically take at least about a few days (e.g., 2, 3, 5, 7, 10 or 14 days) or the symptoms of inflammation reoccur at least once (e.g., once or more times, twice or more times or three or more times). Exemplary causes of chronic inflammation include infections pathogens (e.g., Mycobacterium tuberculosis, protozoa, fungi, and other parasites) that can resist host defenses and remain in the tissue for an extended period, low-level exposure to a material that cannot be eliminated (e.g., silica dust), chronic diseases of the immune system (e.g., rheumatoid arthritis, systemic lupus erythematosus), a defect in inflammation mediating cells (e.g., as in Familial Mediterranean Fever), agents causing oxidative stress and/or mitochondrial dysfunction such as increased production of free radical molecules, advanced glycation end products, uric acid crystals, oxidized lipoproteins, homocysteine (see, e.g., Pahwa et al., 2019, Chronic inflammation).

In some embodiments, the inflammatory disease is characterized by chronic low-grade inflammation, that is, the chronic inflammation symptoms are detectable by laboratory tests (e.g., by measurement of an increased inflammatory marker in the blood) while the subject does not experience clinical inflammatory symptoms for at least one period of time (e.g. for 1, 2, 3, 5, 7, 10 or 14 day(s), 3 weeks, 1, 2, 3, 4, 5, 6, 7, 8, 9, 10 months).

In some embodiments, the inflammatory disease is characterized, classified and/or diagnosed by a certain alteration of an inflammatory marker. In some embodiments, the inflammatory disease is characterized, classified and/or diagnosed by increased CRP, such as, increased CRP in the blood, in a tissue, in an organ and/or in certain cell-types or cells.

In some embodiments, the inflammatory disease is characterized, classified and/or diagnosed by altered immune cell infiltration, activation, proliferation, differentiation, gene expression and/or protein expression in the blood, in a tissue, in an organ and/or in certain cell-types or cells.

Examples of “inflammatory diseases” include myocarditis, inflammatory cardiomyopathy, inflammatory dilated cardiomyopathy, reperfusion injury, allergy, asthma, coeliac disease, glomerulonephritis, hepatitis, inflammatory bowel disease, and transplant rejection among others.

In certain embodiments, the invention relates to an antibody, or an antigen-binding fragment thereof, wherein the binding of the antibody, or the antigen-binding fragment thereof, to Gremlin-1 reduces CD45+ cells in the cardiac tissue and/or decreases systemic inflammation markers in a mouse model for inflammatory cardiomyopathy, particularly wherein (i) the CD45+ cells in the cardiac tissue are myosin specific CD4+ T Cells, and/or (ii) the systemic inflammatory marker is IFN-gamma, IL-17-A and/or CRP.

As described herein, antibodies of the invention can reduce markers of inflammation such as CRP, IFN-γ and IL-17A, reduce immune cell infiltration and reduce accumulation of auto-reactive T-cells in mouse models for inflammatory diseases (e.g., Ex. 4).

Accordingly, the antibody, or the antigen-binding fragment thereof, of the invention, are surprisingly useful for use in treating and/or preventing an inflammatory disease.

In certain embodiments, the invention relates to the antibody, or antigen-binding fragment thereof, for use of the invention, the polynucleotide for use of the invention, or the host cell for use of the invention, or the pharmaceutical composition for use of the invention, wherein the inflammatory disease is an inflammatory disease of the heart.

The term “inflammatory disease of the heart”, as used herein, refers to any inflammatory disease, inflammatory disorder and/or inflammatory condition that affects the heart, preferably affects primarily the heart. Inflammatory diseases of the heart include, inter alia, inflammatory dilated cardiomyopathy, inflammatory cardiomyopathy or myocarditis. In some embodiments the inflammatory disease of the heart is grouped into myocarditis, endocarditis, pericarditis. Causes of inflammatory disease of the heart include without limitation heart attack, kidney failure, rheumatic fever, cancer, medication, infectious agents such as bacterial (e.g., tuberculosis, staphylococci, Escherichia coli or gram-negative organisms such as HACEK), viral (e.g., HIV or Coronaviridae), fungal agents. Inflammatory diseases of the heart include acute and chronic forms of inflammatory diseases of the heart.

As described herein, antibodies of the invention can reduce markers of inflammation such as systemic CRP, IFN-γ and IL-17A in cardiac immune cells, reduce cardiac immune cell infiltration and reduce accumulation of auto-reactive T-cells in cardiac tissue in mouse models for inflammatory diseases (e.g., Ex. 4).

Accordingly, the antibody, or the antigen-binding fragment thereof, of the invention, are surprisingly useful for use in treating and/or preventing an inflammatory disease of the heart.

In certain embodiments, the invention relates to the antibody, or antigen-binding fragment thereof, for use of the invention, the polynucleotide for use of the invention, or the host cell for use of the invention, or the pharmaceutical composition for use of the invention, wherein the inflammatory disease is at least one selected from the group of: inflammatory dilated cardiomyopathy, inflammatory cardiomyopathy, cardiomyopathy, inflammatory cardiomyopathy, myocarditis, pericarditis, perimyocarditis or myopericarditis.

In certain embodiments, the invention relates to the antibody, or antigen-binding fragment thereof, for use of the invention, the polynucleotide for use of the invention, or the host cell for use of the invention, or the pharmaceutical composition for use of the invention, wherein the inflammatory disease is at least one selected from the group of: inflammatory dilated cardiomyopathy, inflammatory cardiomyopathy, pericarditis or myocarditis. The term “inflammatory dilated cardiomyopathy”, as used herein, defines a heterogeneous group of myocardial diseases clinically defined by the presence of left ventricular dilatation and contractile dysfunction, excluding coronary artery disease and myocardial infarction due to their defined etiology. As recognized in the field, myocarditis and inflammatory cardiomyopathy are frequent causes of dilated cardiomyopathy and sudden heart failure.

The term “inflammatory cardiomyopathy”, as used herein, refers to a broad group of disorders characterized by cardiac inflammation; defined by infiltrating immune cells assessed histologically together with signs of cardiac dysfunction, e.g., decreased left ventricle function.

The term “myocarditis”, as used herein, refers to inflammation of the myocardium. In some embodiments myocarditis is determined by histology in endomyocardial biopsy as least 7 CD3⁺ T cells/mm² or >14 leucocytes/mm² including ≤4 CD68⁺ macrophages/mm² or cardiac magnetic resonance with late gadolinium enhancement (LGE) of >4 at baseline.

The ESC working group on myocardial and pericardial disease bases clinical diagnosis of myocarditis and inflammatory cardiomyopathy on the presence of i) ≥1 clinical and ≥1 diagnostic criterion, ii) ≥2 diagnostic criteria, if the patient is asymptomatic.

Clinical presentation: (i) Chest pain, (ii) Acute or chronic heart failure, (iii) Arrhythmic symptoms (palpitations syncope, and sudden cardiac death).

Diagnostic Criteria: (i) Electrocardiogram (ECG) test features (atrioventricular block, bundle branch block, ST/T-wave changes, supraventricular or ventricular arrhythmias, low voltage or QRS complex, and abnormal Q waves); (ii) Markers of myocardial necrosis (elevated cardiac troponins, creatine kinase-MB, NT-proBNP; (iii) Markers of inflammation, preferably CRP; (iv) Functional and structural abnormalities on echocardiography or CMR imaging (impaired left or right ventricle function, with or without left or right ventricle dilation, increased ventricle wall thickness, pericardial effusion, and intracardiac thrombi), (v) Tissue characteristics by CMR with the presence of at least two or three Lake Louise criteria as myocardial edema (indicator of acute inflammation), early gadolinium enhancement (related to hyperemia) and late gadolinium enhancement (related to myocardial necrosis or fibrotic reparative changes).

Myocarditis and inflammatory cardiomyopathy can be subdivided by clinicopathological features (fulminant, acute, chronic active, chronic persistent), autoimmune features (e.g., myocarditis with presence of cardiotoxic T cells and antibodies, eosinophilic myocarditis, giant cell myocarditis, or idiopathic granulomatous myocarditis-related myocarditis), and etiological cause including infectious agents (e.g., viral (enteroviruses (e.g., Coxsackie virus B), erythroviruses (e.g., Parvovirus B19), adenoviruses, or herpes viruses) bacterial (e.g. Corynebacterium diphtheriae, Staphylococcus aureus, Borrelia burgdorferi, and Ehrlichia species) or protozoal (Babesia, or Trypanosoma cruzi) pathogens), exposure to toxic substances (e.g., alcohol, chemicals (hydrocarbons and arsenic), and drugs, including doxorubicin) or exposure to hyperspersensitivity-inducing substances (e.g., sulphonamides or penicillin).

In some embodiments, the antibody, or antigen-binding fragment thereof, of the invention is used in the treatment and/or prevention of a disorder, condition as defined by the ESC Working Group on Myocardial and Pericardial Diseases (e.g., myocarditis and inflammatory cardiomyopathy as defined by the ESC Working Group on Myocardial and Pericardial Diseases).

In some embodiments, myocarditis and/or inflammatory cardiomyopathy is characterized, classified and/or diagnosed based on levels of NT-proBNP >125 mg/ml, >150 mg/ml, >175 mg/ml, >200 mg/ml, >225 mg/ml, >250 mg/ml, >275 mg/ml, or >300 mg/ml. In some embodiments, myocarditis and/or inflammatory cardiomyopathy is characterized, classified and/or diagnosed based on levels of CK >12 u/l, >13 u/l, >14 u/l, >15 u/l. In some embodiments, myocarditis and/or inflammatory cardiomyopathy is characterized, classified and/or diagnosed based on levels of EF<50. In some embodiments, myocarditis and/or inflammatory cardiomyopathy is characterized, classified and/or diagnosed based on a combination and/or ratio of the marker levels described herein.

In some embodiments, the antibody, or antigen-binding fragment thereof, of the invention is used in the treatment and/or prevention of autoimmune myocarditis, e.g., mediated by MYH6-specific T cells. In some embodiments, the antibody, or antigen-binding fragment thereof, of the invention is used in the treatment and/or prevention of infectious myocarditis, e.g. induced by Coxsackie B virus infection. In some embodiments, the antibody, or antigen-binding fragment thereof, of the invention is used in the treatment and/or prevention of acute myocarditis. In some embodiments, the antibody, or antigen-binding fragment thereof, of the invention is used in the treatment and/or prevention of chronic active myocarditis and/or chronic active inflammatory cardiomyopathy. In some embodiments, the antibody, or antigen-binding fragment thereof, of the invention is used in the treatment and/or prevention of chronic persistent inflammatory cardiomyopathy.

As described herein, antibodies of the invention can reduce markers of inflammation such as systemic CRP, IFN-γ and IL-17A in cardiac immune cells, reduce cardiac immune cell infiltration and reduce accumulation of auto-reactive cardiotropic MYH6-specific CD4+ T cells in mouse models for inflammatory diseases (e.g., Ex. 4).

Accordingly, the antibody, or the antigen-binding fragment thereof, of the invention, are surprisingly useful for use in treating and/or preventing inflammatory dilated cardiomyopathy, inflammatory cardiomyopathy, pericarditis or myocarditis.

In certain embodiments, the invention relates to the antibody, or antigen-binding fragment thereof, for use of the invention, the polynucleotide for use of the invention, or the host cell for use of the invention, or the pharmaceutical composition for use of the invention, wherein the inflammatory disease is pericarditis.

In certain embodiments, the invention relates to the antibody, or antigen-binding fragment thereof, for use of the invention, the polynucleotide for use of the invention, or the host cell for use of the invention, or the pharmaceutical composition for use of the invention, wherein the inflammatory disease is perimyocarditis and/or myopericarditis.

Acute pericarditis is typically diagnosed based on two of the following criteria: chest pain, pericardial rubbing, typical changes in the electrocardiogram, with new and widespread ST elevation or PR depression in the acute phase, and pericardial effusion. Increased CRP levels frequently confirm pericarditis diagnosis. Many patients present with an acute inflammation of the pericardium and the underlying myocardium; a condition that is referred to as myopericarditis. Myopericarditis patients show primarily pericarditis symptoms with involvement of the myocardium diagnosed by cardiac biomarker elevation or imaging modalities. The diagnosis of perimyocarditis is mainly used for conditions with evidence of regional wall motion abnormalities, but reduced ventricular function, (Imazio, Massimo, and Rita Trinchero. International journal of cardiology vol. 127, 1 (2008): 17-26.; Adler, Yehuda et al. European heart journal vol. 36, 42 (2015): 2921-2964). Notably the pathological mechanisms underlying both, perimyocarditis and myopericarditis were underexplored, which had led to inaccurate epidemiological information and more importantly to the lack of specific treatments for patients that present with these diseases.

In certain embodiments, the invention relates to the antibody, or antigen-binding fragment thereof, for use of the invention, the polynucleotide for use of the invention, or the host cell for use of the invention, or the pharmaceutical composition for use of the invention wherein the inflammatory disease is reperfusion injury, allergy, asthma, coeliac disease, glomerulonephritis, hepatitis, inflammatory bowel disease or transplant rejection.

In certain embodiments, the antibody, or antigen-binding fragment thereof, of the invention is used for treating and/or preventing reperfusion injury.

The term “reperfusion injury”, as used herein, relates to organ or tissue damage caused when blood supply returns to the organ or tissue after a period of ischemia. The absence of oxygen and nutrients from blood during the ischemic period creates a condition in which the restoration of circulation results in inflammation and oxidative damage through the induction of oxidative stress rather than restoration of normal function. Oxidative stress associated with reperfusion may cause damage to the affected tissues or organs. Reperfusion injury is characterized biochemically by a depletion of oxygen during an ischemic event followed by reoxygenation and the concomitant generation of reactive oxygen species during reperfusion. The injury that occurs with reperfusion is the result of the interaction between the substances that accumulate during ischemia and those that are delivered on reperfusion. The cornerstone of these events is oxidative stress, defined as the imbalance between oxygen radicals and the endogenous scavenging system. The result is cell injury and death, which is initially localized, but eventually becomes systemic if the inflammatory reaction is unchecked. Reperfusion injury may result, inter alia, in organ dysfunction (in the ischemic organ or in any other organ), infarct, inflammation (in the damaged organ or tissue), oxidative damage, mitochondrial membrane potential damage, apoptosis, reperfusion-related arrhythmia, cardiac stunning, cardiac lipotoxicity, ischemia-derived scar formation, and combinations thereof.

As described herein, antibodies of the invention can reduce markers of inflammation such as CRP, IFN-γ and IL-17A, reduce immune cell infiltration and reduce accumulation of auto-reactive T-cells in mouse models for inflammatory diseases (e.g., Ex. 4).

Accordingly, the antibody, or the antigen-binding fragment thereof, of the invention, are surprisingly useful for use in treating and/or preventing a reperfusion injury.

In certain embodiments, the antibody, or antigen-binding fragment thereof, of the invention is used for treating and/or preventing allergy.

The term “allergy”, as used herein, refers to a state of immune responsiveness in a subject specific to an exogenous antigen (or “allergen”) that is not otherwise harmful to the subject. Symptoms of allergy may include generalized phenomena such as inflammation, respiratory complaints, swelling, or distress typically associated with allergy, rhinitis, edema, and allergic skin disorders including but not limited to atopic dermatitis (e.g., eczema), urticaria (e.g., hives) and angioedema, and allergic contact dermatitis. More specific phenomena that are “symptoms” of an allergic response include any measurable or observable change, for example at the cellular level, including but not limited to local or systemic changes in cell populations, eosinophilia, recruitment and/or activation of immune cells, including, for example, mast cells and/or basophils, changes in antigen-presenting cells (including but not limited to FcεRI-bearing dendritic cells), intracellular or molecular changes, including measurement or observations of one or more steps in an immunological cascade, release of intracellular compounds that mediate an allergic response (e.g., mediators), and changes in one or more cytokines (e.g., IL-3, IL-5, IL-9, IL-4, or IL-13) or related compounds or antagonists thereof.

Traditional treatments of allergy have CNS-side effects due to blood-brain barrier permeability (Lieberman P., 2009, Allergy Asthma Proc. 30(5):482-6). In some embodiments, the antibody, or antigen-binding fragment thereof, of the invention has limited blood-brain barrier permeability.

As described herein, antibodies of the invention can reduce markers of inflammation such as CRP, IFN-γ and IL-17A, reduce immune cell infiltration and reduce accumulation of auto-reactive T-cells in mouse models for inflammatory diseases (e.g., Ex. 4).

The anti-inflammatory properties and/or the limited blood-brain barrier permeability of the antibody, or antigen-binding fragment thereof, of the invention are useful in the treatment of allergy. Furthermore, the high Gremlin-1 activity reduction capacity of the antibody, or antigen-binding fragment thereof, of the invention enables low effective doses that are particularly useful (e.g., long administration intervals, few (class-)side effects) for in a chronic disease such as allergy.

Accordingly, the antibody, or the antigen-binding fragment thereof, of the invention, are surprisingly useful for use in treating and/or preventing an allergy.

In certain embodiments, the antibody, or antigen-binding fragment thereof, of the invention is used for treating and/or preventing asthma.

The term “asthma”, as used herein, refers to a disorder of the respiratory system characterized by inflammation, narrowing of the airways, and/or increased reactivity of the airways to inhaled agents. Asthma is frequently, although not exclusively, associated with atopic or allergic symptoms. The effects of asthma, which are reversed or prevented by the method of the present invention include, for example, airway hyperresponsiveness to one or more environmental or other allergens, airway inflammation, airway obstruction, tissue and/or blood eosinophilia, and/or mucus hypersecretion. The effects of asthma can be evaluated clinically, cellularly, serologically, or by any other suitable method. Illustratively, the asthmatic response can, in some cases, be characterized as a type I hypersensitivity reaction. This can involve allergen-specific immunoglobulins of the IgE class bound to high-affinity receptors on the surfaces of mast cells present in the sub-epithelial layer of the airways. Cross-linking of these bound IgE molecules results in an immediate release of mediators, including leukotrienes, prostaglandins and histamine, which are capable of contracting airway smooth muscle cells and induce edema and mucus secretion leading to narrowed, spastic airways. Although airborne allergens are common triggers of these attacks in allergic asthmatics, other agents (such as cold air, lower respiratory tract infections, and stress) can also stimulate attacks. In addition to the immediate release of bronchospastic mediators, cytokines and chemokines can be locally produced. Chemokines stimulate the recruitment of eosinophils, macrophages, neutrophils, and T lymphocytes. Once present, effector cells, such as eosinophils, may be prompted to release a collection of toxic granules. These granules may cause further, prolonged bronchoconstriction and damage epithelial layers. This damage, coupled with profibrotic cytokines also released by eosinophils and epithelial cells, can lay the groundwork for the process of airway remodeling to begin. Further, cytokines released at the time of mast cell degranulation can have more global effects. These include the recruitment of eosinophils from bone marrow and peripheral sources in addition to encouraging their survival (primarily via IL-5 and GM-CSF) and the stimulation and continued production of IgE by B-cells as well as the induction of vascular cell adhesion molecule-1 (“VCAM-1”) by endothelial cells (IL-4). Moreover, cytokines, such as IL-4 and IL-5, can have the effect of ensuring that this cycle of allergic inflammation persists. The method of the present invention can be used to prevent or reverse some or all of these effects of asthma.

Some treatments for asthma have CNS-side effects due to blood-brain barrier permeability (Lieberman P., 2009, Allergy Asthma Proc. 30(5):482-6). In some embodiments, the antibody, or antigen-binding fragment thereof, of the invention has limited blood-brain barrier permeability.

Some treatments for asthma impair oral health (e.g., increase risk for yeast infection) (see, e.g., Godara, Navneet et al., 2011, Lung India: official organ of Indian Chest Society vol. 28, 4: 272-5). In some embodiments, the antibody, or antigen-binding fragment thereof, of the invention has a selective effect the immune system and has no effect on host defense or a limited effect on host defense. Therefore, in some embodiments, the antibody, or antigen-binding fragment thereof, of the invention is used in the treatment for asthma and does not impair oral health.

In some embodiments, the antibody, or antigen-binding fragment thereof, of the invention has a local effect (e.g. in the nose and/or in the lung) and a limited systemic absorption, e.g., after inhalation, intrapulmonary administration and/or intranasal application.

As described herein, antibodies of the invention can reduce markers of inflammation such as CRP, IFN-γ and IL-17A, reduce immune cell infiltration and reduce accumulation of auto-reactive T-cells in mouse models for inflammatory diseases (e.g., Ex. 4).

The anti-inflammatory properties and/or the selective local action of the antibody, or antigen-binding fragment thereof, of the invention are useful in the treatment of asthma. Furthermore, the high Gremlin-1 activity reduction capacity of the antibody, or antigen-binding fragment thereof, of the invention enables low effective doses that are particularly useful (e.g., long administration intervals, few (class-)side effects) for in a chronic disease such as asthma.

Accordingly, the antibody, or the antigen-binding fragment thereof, of the invention, are surprisingly useful for use in treating and/or preventing asthma.

In certain embodiments, the antibody, or antigen-binding fragment thereof, of the invention is used for treating and/or preventing glomerulonephritis.

The term “glomerulonephritis”, as used herein, refers to a renal disease characterized by inflammation of the glomeruli, or small blood vessels in the kidneys. It may present with isolated hematuria and/or proteinuria or as a nephrotic syndrome, acute renal failure, or chronic renal failure. Glomerulonephritis is categorized into several different pathological patterns, which may be grouped into non-proliferative or proliferative types.

As described herein, antibodies of the invention can reduce markers of inflammation such as CRP, IFN-γ and IL-17A, reduce immune cell infiltration and reduce accumulation of auto-reactive T-cells in mouse models for inflammatory diseases (e.g., Ex. 4).

Accordingly, the antibody, or the antigen-binding fragment thereof, of the invention, are surprisingly useful for use in treating and/or preventing glomerulonephritis.

In certain embodiments, the antibody, or antigen-binding fragment thereof, of the invention is used for treating and/or preventing hepatitis.

The term “hepatitis”, as used herein, refers to any inflammatory disease, inflammatory disorder or inflammatory condition that affects the liver tissue. Diagnosis of hepatitis may be made on the basis of some or all of the following: a person's signs and symptoms, medical history, blood tests, imaging, and liver biopsy. For some forms of hepatitis, a person's blood test and clinical picture can be sufficient for diagnosis. Hepatitis includes, without limitation, viral hepatitis, steatohepatitis and cirrhosis. Causes of hepatitis include, without limitation, infections (e.g., viruses), alcohol use, medications, toxins, diseases of the immune system, and non-alcoholic steatohepatitis. Viral hepatitis includes, without limitation, hepatitis A, hepatitis B, hepatitis C, hepatitis D, hepatitis E. Steatohepatitis includes, without limitation, alcoholic steatohepatitis and non-alcoholic steatohepatitis. In some embodiments hepatitis refers to acute hepatitis and/or fulminant hepatitis, in other embodiments hepatitis refers to chronic hepatitis.

In some embodiments, the antibody, or antigen-binding fragment thereof, of the invention reduces and/or prevents formation of fibrosis.

As described herein, antibodies of the invention can reduce markers of inflammation such as CRP, IFN-γ and IL-17A, reduce immune cell infiltration and reduce accumulation of auto-reactive T-cells in mouse models for inflammatory diseases (e.g., Ex. 4).

The anti-inflammatory properties of the antibody, or antigen-binding fragment thereof, of the invention are useful in the treatment of hepatitis. Furthermore, the high Gremlin-1 activity reduction capacity of the antibody, or antigen-binding fragment thereof, of the invention enables low effective doses that are particularly useful (e.g., long administration intervals, few (class-)side effects) for in a hepatitis, particularly in chronic forms of hepatitis.

Accordingly, the antibody, or the antigen-binding fragment thereof, of the invention, are surprisingly useful for use in treating and/or preventing hepatitis.

In certain embodiments, the antibody, or antigen-binding fragment thereof, of the invention is used for treating and/or preventing inflammatory bowel disease.

The term “inflammatory bowel disease”, as used herein, refers to any inflammatory disease, inflammatory disorder or inflammatory condition that affects the bowel. The term “inflammatory bowel disease” includes but is not limited to ulcerative colitis, Crohn's disease, especially Crohn's disease in a state that affect specifically the colon with or without ileitis, microscopic colitis (lymphocytic colitis and collagenous colitis), infectious colitis caused by bacteria or by virus, radiation colitis, ischemic colitis, pediatric colitis, undetermined colitis, and functional bowel disorders (described symptoms without evident anatomical abnormalities).

In some embodiments, the antibody, or antigen-binding fragment thereof, of the invention has a certain stability in the gastrointestinal environment (e.g., as described Virdi V et al., 2019, Nat Biotechnol., 37(5):527-530) and can be administered enterally, orally and/or rectally. In certain embodiments, the antibody, or antigen-binding fragment thereof, of the invention induces no substantial systemic blood concentration increase of the antibody, or antigen-binding fragment thereof, of the invention after local (e.g., enteral) administration.

As described herein, antibodies of the invention can reduce markers of inflammation such as CRP, IFN-γ and IL-17A, reduce immune cell infiltration and reduce accumulation of auto-reactive T-cells in mouse models for inflammatory diseases (e.g., Ex. 4).

The anti-inflammatory properties and/or the local action of the antibody, or antigen-binding fragment thereof, of the invention are useful in the treatment of inflammatory bowel disease. Furthermore, the high Gremlin-1 activity reduction capacity of the antibody, or antigen-binding fragment thereof, of the invention enables low effective doses that are particularly useful (e.g., long administration intervals, few (class-)side effects) for in a chronic disease such as inflammatory bowel disease.

Accordingly, the antibody, or the antigen-binding fragment thereof, of the invention, are surprisingly useful for use in treating and/or preventing inflammatory bowel disease.

In certain embodiments, the antibody, or antigen-binding fragment thereof, of the invention is used for treating and/or preventing coeliac disease.

The term “celiac disease”, as used herein, refers to an inflammatory disease of the small intestine caused by the ingestion of gluten proteins from widely prevalent food sources such as wheat. Celiac disease includes, inter alia, clinically silent celiac disease, characterized by absence of gastrointestinal symptoms, and moderate to severe symptomatic celiac disease, characterized by gastrointestinal symptoms that can range from mild to severe. “Celiac disease” as used herein also includes dermatitis herpetiformis.

As described herein, antibodies of the invention can reduce markers of inflammation such as CRP, IFN-γ and IL-17A, reduce immune cell infiltration and reduce accumulation of auto-reactive T-cells in mouse models for inflammatory diseases (e.g., Ex. 4).

The anti-inflammatory properties and/or the local action of the antibody, or antigen-binding fragment thereof, of the invention are useful in the treatment of coeliac disease. Furthermore, the high Gremlin-1 activity reduction capacity of the antibody, or antigen-binding fragment thereof, of the invention enables low effective doses that are particularly useful (e.g., long administration intervals, few (class-)side effects) for in a chronic disease such as coeliac disease.

Accordingly, the antibody, or the antigen-binding fragment thereof, of the invention, are surprisingly useful for use in treating and/or preventing celiac disease.

In certain embodiments, the antibody, or antigen-binding fragment thereof, of the invention is used for treating and/or preventing transplant rejection.

The term “transplant rejection”, as used herein, refers to a consequence of cell, tissue or organ transplantation caused by an inflammatory response of the recipient's or host's immune system in response to the transplanted cell/tissue/organ, which can damage or destroy the transplanted cell/tissue/organ.

As described herein, antibodies of the invention can reduce markers of inflammation such as CRP, IFN-γ and IL-17A, reduce immune cell infiltration and reduce accumulation of auto-reactive T-cells in mouse models for inflammatory diseases (e.g., Ex. 4).

The anti-inflammatory properties, the selective and/or the local action of the antibody, or antigen-binding fragment thereof, of the invention are useful in the treatment of transplant rejection. Furthermore, the high Gremlin-1 activity reduction capacity of the antibody, or antigen-binding fragment thereof, of the invention enables low effective doses that are particularly useful (e.g., long administration intervals, few (class-)side effects) for in a chronic disease such as transplant rejection.

Accordingly, the antibody, or the antigen-binding fragment thereof, of the invention, are surprisingly useful for use in treating and/or preventing transplant rejection.

In certain embodiments, the invention relates to the antibody, or antigen-binding fragment thereof, of the invention, the polynucleotide of the invention or the host cell of the invention, or the pharmaceutical composition of the invention for use in the treatment and/or prevention of a disease or disorder associated with SARS-CoV-2 infection and/or SARS-CoV-2 vaccination.

The term “disease or disorder associated with SARS-CoV-2 infection and/or SARS-CoV-2 vaccination”, as used herein, refers to any disease or disorder that occurs in patients with a history of SARS-CoV-2 infection and/or SARS-CoV-2 vaccination. A “disease or disorder associated with SARS-CoV-2 infection and/or SARS-CoV-2 vaccination” also encompasses symptoms of diseases or disorders associated with SARS-CoV-2 infection and/or SARS-CoV-2 vaccination, as well as symptoms of a SARS-CoV-2 infection and/or SARS-CoV-2 vaccination itself. The term “patients with a history of SARS-CoV-2 infection”, as used herein, refers to patients with at least one selected from the group consisting of: contact to a SARS-CoV-2 positive person, self-reported history of SARS-CoV-2 symptoms, antibodies against SARS-CoV-2 and a history of at least one positive SARS-CoV-2 test.

In some embodiments, the patient with a history of SARS-CoV-2 infection, described herein, are patients with a history of a positive SARS-CoV-2 test, preferably a positive SARS-CoV-2 PCR test.

In some embodiments, the patient with a history of SARS-CoV-2 infection had within the last 22, 21, 20, 19, 18, 17, 18, 19, 18, 17, 16, 15, 14, 13, 12, 11, 10, 9, 8, 7, 6, 5, 4, 3, 2, 1 year(s), 11, 10, 9, 8, 7, 6, 5, 4, 3, 2, 1 month(s), 4, 3, 2 or 1 week(s) at least one selected from the group consisting of: contact to a SARS-CoV-2 positive person, self-reported SARS-CoV-2 symptoms, detectable antibodies against SARS-CoV-2 and a positive SARS-CoV-2 positive test, preferably a positive SARS-CoV-2 PCR test.

In some embodiments, the SARS-CoV-2 vaccine described herein is at least one SARS-CoV-2 vaccine selected from the group consisting of: Pfizer-BioNTech, Moderna, ZyCoV-D, Oxford-AstraZeneca, Janssen, Sputnik V, Sputnik Light, Convidecia, Sinopharm BIBP, CoronaVac, Covaxin, Sinopharm WIBP, CoviVac, Novavax, Abdala, EpiVacCorona, Zifivax and Soberana 02.

In some embodiments, the SARS-CoV-2 vaccine described herein is a subunit vaccine, an mRNA vaccine and/or an adenovirus vector vaccine.

In some embodiments, the disease or disorder associated with SARS-CoV-2 infection and/or SARS-CoV-2 vaccination described herein is a cytokine-mediated disease or disorder.

In some embodiments, the disease or disorder associated with SARS-CoV-2 infection and/or SARS-CoV-2 vaccination is a disease or disorder affecting the heart function.

In certain embodiments, the invention relates to the antibody, or antigen-binding fragment thereof, for use of the invention, the polynucleotide for use of the invention, or the host cell for use of the invention, or the pharmaceutical composition for use of the invention, wherein the disease or disorder associated with SARS-CoV-2 infection and/or SARS-CoV-2 vaccination is an inflammatory disease of the heart.

In certain embodiments, the invention relates to the antibody, or antigen-binding fragment thereof, for use of the invention, the polynucleotide for use of the invention, or the host cell for use of the invention, or the pharmaceutical composition for use of the invention, wherein the disease or disorder associated with SARS-CoV-2 infection and/or SARS-CoV-2 vaccination is heart failure.

In certain embodiments, the invention relates to the antibody, or antigen-binding fragment thereof, for use of the invention, the polynucleotide for use of the invention, or the host cell for use of the invention, or the pharmaceutical composition for use of the invention, wherein the disease or disorder associated with SARS-CoV-2 infection and/or SARS-CoV-2 vaccination is heart failure and an inflammatory disease of the heart.

Each embodiment described herein may be combined with any other embodiment described herein.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The general methods and techniques described herein may be performed according to conventional methods well known in the art and as described in various general and more specific references that are cited and discussed throughout the present specification unless otherwise indicated.

While aspects of the invention are illustrated and described in detail in the figures and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Although the foregoing invention has been described in some detail by way of illustration and example for purposes of clarity of understanding, it will be obvious that certain changes and modifications may be practiced within the scope of the appended claims.

### BRIEF DESCRIPTION OF FIGURES

FIG. 1A shows the design of sixteen humanized variants based on the in silico analyses of parental mouse panGREM antibody 14-D10-2.

FIG. 1B shows an SDS-PAGE analysis of the purified chimeric antibody control (chimeric) and sixteen humanized antibodies (Var_1 to Var 16).

FIG. 2A shows specific binding of chimeric antibody control and sixteen humanized antibodies (Var 1 to Var 16) to human Gremlin-1 (huGREM1) and human Gremlin-2 (huGREM2) determined by ELISA.

FIG. 2B shows the binding of chimeric antibody control and five humanized antibodies (Var 3, Var 5, Var 7, Var_11 and Var 13) to the shared epitopes GREM1₁₀₅₋₁₂₁ and GREM2₈₄₋₁₀₀.

FIG. 3A illustrates the production of alkaline phosphatase (ALP) in the presence of BMP4 alone (high ALP production), after addition of different concentrations of huGREM1 protein to inhibit BMP4 binding (low ALP production) and after addition of the humanized antibodies (Var 5, Var_7 and Var_13) to characterize their neutralizing capacity to specifically prevent huGREM1-mediated BMP4 inhibition.

FIG. 3B illustrates the production of alkaline phosphatase (ALP) in the presence of BMP4 alone (high ALP production), after addition of different concentrations of huGREM2 protein to inhibit BMP4 binding (low ALP production) and after addition of the humanized antibodies (Var 5, Var_7 and Var_13) to characterize their neutralizing capacity to specifically prevent huGREM2-mediated BMP4 inhibition.

FIG. 4A illustrates the experimental setup in which Rag1^{-/-} BALB/c mice receiving cardiac myosin-specific CD4⁺ T cells are treated with either 200 μg of isotype control antibody, 14-D10-2, or humanized variants Var_5 or Var_7 twice per week as indicated. Immune cell infiltration in the heart was analyzed on day 28.

FIG. 4B shows the quantification of heart-infiltrating CD45⁺ immune cells in the myocardium of Rag1^{-/-} recipient mice prophylactically treated with the indicated antibodies.

FIG. 4C shows the quantification of cardiac myosin-specific CD4⁺ T cells in the hearts of Rag1^{-/-} recipient mice prophylactically treated with the indicated antibodies.

FIG. 4D shows the quantification of IFN-γ and IL-17A cytokine-expressing, cardiac myosin-specific CD4⁺ T cells in the heart. The values are pooled from 2 independent experiments with N=7-8 mice per group.

FIGS. 5A and B show SDS-PAGE analysis of the purified, humanized antibodies (Var_5 and Var_7) after incubation at 25° C. (A) or 37° C. (B) for 1, 7 or 14 days.

FIG. 6A Shows the concentration of human IgG4 in the serum of Rag1^{-/-} mice that received 200 μg of chimeric control antibody or Var_5 or Var_7. Antibody concentration was measured at the indicated days post administration. The values are pooled from 2 independent experiments with N=5 mice per group.

FIG. 6B Illustrates the in vivo availability of the humanized 14-D10-2 variants Var_5 and Var_7 compared to the chimeric antibody control, based on the area under the curve (AUC) calculation.

An “effective amount” of an agent, e.g., a therapeutic agent, refers to an amount effective, at dosages and for periods of time necessary, to achieve the desired therapeutic or prophylactic result. Furthermore, the effective amount may depend on the individual patient's history, age, weight, family history, genetic makeup (e.g. HLA genotype), stage of myocarditis, the types of preceding or concomitant treatments, if any, and other individual characteristics of the subject to be treated.

As used herein as “subject” is an animal, such as a mammal, including a primate (such as a human a non-human primate, e.g. a monkey, and a chimpanzee), a non-primate (such as a cow a pig, a camel, a llama, a horse, a goat, a rabbit, a sheep, a hamster, a guinea pig, a cat, a dog, a rat, a mouse, a horse and a whale), or a bird (e.g. a duck or a goose).

“a,” “an,” and “the” are used herein to refer to one or to more than one (i.e., to at least one, or to one or more) of the grammatical object of the article.

“or” should be understood to mean either one, both, or any combination thereof of the alternatives.

“and/or” should be understood to mean either one, or both of the alternatives.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

The terms “include” and “comprise” are used synonymously. “Preferably” means one option out of a series of options not excluding other options. “E.g.” means one example without restriction to the mentioned example. By “consisting of” is meant including, and limited to, whatever follows the phrase “consisting of.” The terms “about” or “approximately”, as used herein, refer to “within 20%”, more preferably “within 10%”, and even more preferably “within 5%”, of a given value or range.

The term “synergistic”, as used herein, means that the effect achieved in a combination is greater than the sum of the effects that result from the active ingredients of the combination separately.

The term “anti-Gremlin-1 antibody”, as used herein, refers to an antibody, or antigen-binding fragment thereof, binding to Gremlin-1 wherein the binding to Gremlin-1 reduces Gremlin-1 activity. In a preferred embodiment, the anti-Gremlin-1 antibody binds specifically to Germlin-1.

The term “monoclonal antibody”, as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Monoclonal antibodies are advantageous in that they may be synthesized by a hybridoma culture, essentially uncontaminated by other immunoglobulins. The modified “monoclonal” indicates the character of the antibody as being amongst a substantially homogeneous population of antibodies and is not to be construed as requiring production of the antibody by any particular method. As mentioned above, the monoclonal antibodies to be used in accordance with the present invention may be made by the hybridoma method described by Kohler, G. et al., 1975, Nature 256.5517: 495-497.

The term “polyclonal antibody”, as used herein, refers to an antibody which was produced among or in the presence of one or more other, non-identical antibodies. In general, polyclonal antibodies are produced from a B lymphocyte in the presence of several other B-lymphocytes which produced non-identical antibodies. Usually, polyclonal antibodies are obtained directly from an immunized animal.

The term “fully-human antibody”, as used herein, refers to an antibody, which comprises human immunoglobulin protein sequences only. A fully human antibody may contain murine carbohydrate chains if produced in a mouse, in a mouse cell or in a hybridoma derived from a mouse cell. Similarly, “mouse antibody” or “murine antibody” refers to an antibody, which comprises mouse/murine immunoglobulin protein sequences only. Alternatively, a “fully-human antibody” may contain rat carbohydrate chains if produced in a rat, in a rat cell, in a hybridoma derived from a rat cell. Similarly, the term “rat antibody”, as used herein, refers to an antibody that comprises rat immunoglobulin sequences only. Fully-human antibodies may also be produced, for example, by phage display, which is a widely used screening technology which enables production and screening of fully human antibodies. In alternative embodiments, phage antibodies can be used in context of this invention. Phage display methods are described, for example, in U.S. Pat. Nos. 5,403,484, 5,969,108 and 5,885,793. Another technology which enables development of fully-human antibodies involves a modification of mouse hybridoma technology. Mice are made transgenic to contain the human immunoglobulin locus in exchange for their own mouse genes (see, for example, U.S. Pat. No. 5,877,397).

“Humanized” forms of non-human (e.g. murine or rabbit) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Often, humanized antibodies are human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Furthermore, humanized antibody may comprise residues, which are found neither in the recipient antibody nor in the imported CDR or framework sequences. These modifications are made to further refine and optimize antibody performance. In general, the humanized antibody will comprise substantially all of at least one, and typically two variable domains, in which all or substantially all of the CDR regions correspond to those of a non-human immunoglobulin and all or substantially all of the FR regions are those of a human immunoglobulin consensus sequence. The humanized antibody may also comprise at least a portion of an immunoglobulin constant region (Fc), typically that of a human immunoglobulin. For further details, see: Jones et al., 1986, Nature 321, 522-525; Reichmann et al., 1998, Nature 332, 323-327 and Presta et al., 1992, Curr Op Struct Biol 2, 593-596.

A popular method for humanization of antibodies involves CDR grafting, where a functional antigen-binding site from a non-human ‘donor’ antibody is grafted onto a human ‘acceptor’ antibody. CDR grafting methods are known in the art and described, for example, in U.S. Pat. Nos. 5,225,539, 5,693,761 and 6,407,213. Another related method is the production of humanized antibodies from transgenic animals that are genetically engineered to contain one or more humanized immunoglobulin loci which are capable of undergoing gene rearrangement and gene conversion (see, for example, U.S. Pat. No. 7,129,084).

The term “chimeric antibodies”, refers to an antibody, which comprises a variable region of the present invention fused or chimerized with an antibody region (e.g., constant region) from another, human or non-human species (e.g., mouse, horse, rabbit, dog, cow, chicken).

The term “recombinant (human) antibody” includes all human sequence antibodies that are prepared, expressed, created or isolated by recombinant means, such as antibodies isolated from an animal (e.g., a mouse) that is transgenic for human immunoglobulin genes; antibodies expressed using a recombinant expression vector transfected into a host cell, antibodies isolated from a recombinant, combinatorial human antibody library, or antibodies prepared, expressed, created or isolated by any other means that involves splicing of human immunoglobulin gene sequences to other DNA sequences. Such recombinant human antibodies have variable and constant regions (if present) derived from human germline immunoglobulin sequences. Such antibodies can, however, be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in VIVO.

A “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

The term “heterohybrid antibody”, as used herein, refers to an antibody having light and heavy chains of different organismal origins. For example, an antibody having a human heavy chain associated with a murine light chain is a heterohybrid antibody. Examples of heterohybrid antibodies include chimeric and humanized antibodies.

The term “isotype”, as used herein, refers to the antibody class (e.g., IgM or IgG1) that is encoded by heavy chain constant region genes.

The term “vector”, as used herein, refers to a nucleic acid molecule, capable transferring or transporting another nucleic acid molecule. The transferred nucleic acid is generally linked to, i.e., inserted into, the vector nucleic acid molecule. A vector may include sequences that direct autonomous replication in a cell or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses.

The term “viral vector”, as used herein, refers either to a virus or viral particle capable of transferring a nucleic acid into a cell or to the transferred nucleic acid itself. Viral vectors and transfer plasmids contain structural and/or functional genetic elements that are primarily derived from a virus. The term “viral vector” includes, inter alia, the viral vectors described by Lundstrom, Kenneth, 2018, Diseases vol. 6, 2 42.

The term “antigenic peptide”, as used herein, refers to a peptide, which is recognized by the host immune system. In some embodiments the antigenic peptide is prone to induce/elicit, increase, prolong and/or maintain an immune response in a subject to whom it is administered. In some embodiments the antigenic peptide is prone to induce/elicit, increase, prolong and/or maintain an immune tolerance towards an agent (e.g. the antibody, or the antigen-binding fragment thereof, of the invention) in a subject to whom it is administered.

Reference throughout this specification to “one embodiment,” “an embodiment,” “a particular embodiment,” “a related embodiment,” “a certain embodiment,” “an additional embodiment,” “a specific embodiment” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

The invention will be further described in the following examples, which do not limit the scope of the invention described in the claims.

### EXAMPLES

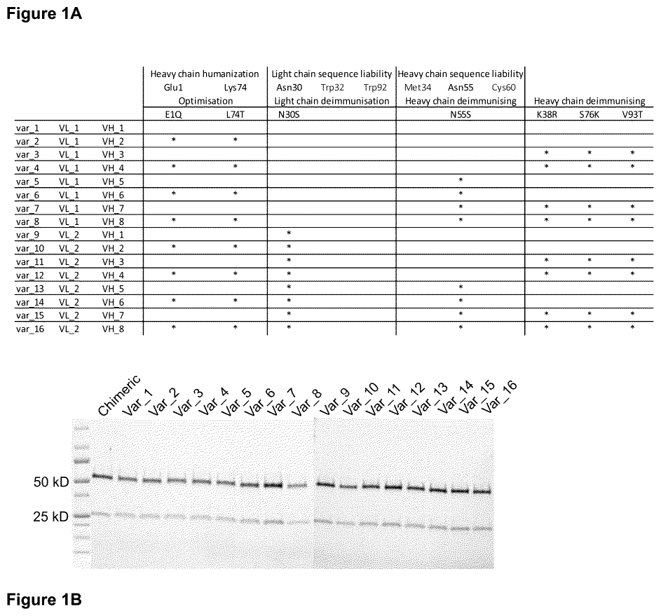

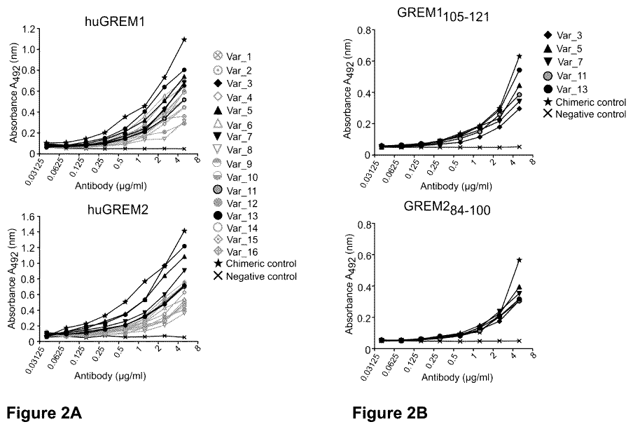

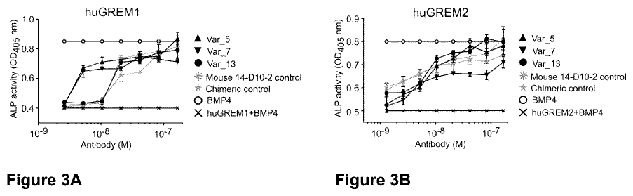

The inventors investigated humanization of the anti-GREM1/2 mouse antibody 14-D10-2 (having a HC comprising SEQ ID NO: 31 and a LC comprising SEQ ID NO: 33). Based on the in silico analysis algorithm, Epibase® and in silico Manufacturability Assessment program, and designed sixteen humanized antibody variants with different substitutions of amino acids in the heavy and light chains to facilitate the humanization process including removal of potential sequence liabilities and de-immunization (FIG. 1A). It is noteworthy that the substitutions of asparagine to serine (N30S) in the light chain and asparagine to serine (N55S) in the heavy chain are situated in the CDR regions, while other substitutions are situated in the immunoglobulin framework (FIG. 1A). Substitution of these amino acids are predicted to not only remove the potential sequence liability for manufacturability and stability of humanized antibody, but also to reduce the potential immunogenicity. In total, sixteen humanized variants with different amino acid substitutions have been designed as detailed FIG. 1A. All sixteen humanized 14-D10-2 variants were generated using the Lonza Biologics GS Xceed™ gene expression system and humanized antibodies were purified using protein A columns (FIG. 1B). Five out of sixteen humanized 14-D10-2 variants (Var_3 (having a HC comprising SEQ ID NO: 11 and a LC comprising SEQ ID NO: 16), Var_5 (having a HC comprising SEQ ID NO: 12 and a LC comprising SEQ ID NO: 17), Var_7 (having a HC comprising SEQ ID NO: 13 and a LC comprising SEQ ID NO: 18), Var 11 (having a HC comprising SEQ ID NO: 14 and a LC comprising SEQ ID NO: 19) and Var_13 (having a HC comprising SEQ ID NO: 15 and a LC comprising SEQ ID NO: 20)) exhibited high binding activities against both huGREM1 and huGREM2 proteins (FIG. 2A). In addition, the selected five humanized antibody variants bind to the cross-reactive GREM epitopes GREM1₁₀₅₋₁₂₁ and GREM2₈₄₋₁₀₀ (FIG. 2B). The sequences of the humanized 14-D10-2 variants are listed in Table 1. The ability of the selected five humanized 14-D10-2 variants to neutralize huGREM1 and huGREM2 activity was tested in ATDC5 cells that produce alkaline phosphatase response to BMP4 stimulation. Addition of humanized antibody variants in this assay revealed that Var_5 and Var_7 possess superior neutralization activity against huGREM1 when compared to the chimeric antibody control, which contains the full-length murine 14-D10-2 heavy chain and light chain and the human IgG4 constant chain, or the parental mouse 14-D10-2 antibody (FIG. 3A). In addition, huGREM2 activity was neutralized by the humanized 14-D10-2 variants (FIG. 3B).

In sum, humanized anti-GREM1/2 variants bind to both huGREM1 and huGREM2 and neutralize the activity of both huGREM1 and huGREM2 through the substitution of amino acids in CDRs of the light and heavy chains.

### Example 1: Generation of panGREM Antibody Humanized Variants

### 1. In Silico Analyses for Antibody Humanization

The amino acid sequences of heavy chain and light chain from panGREM mouse antibody 14-D10-2 were utilized for the antibody humanization through Epibase® and in silico tools provided by Lonza. Sequences were analyzed by the Conserved Domain Database (Marchler-Bauer, Aron et al. Nucleic acids research vol. 39, Database issue (2011): D225-9) to identify the domain content of each amino acid including the CDR regions and the amino acid residues in critical positions. Next, these sequences were aligned to a set of human genome reference sequences using MAFFT (Katoh, Kazutaka et al. Nucleic acids research vol. 30, 14 (2002): 3059-66.) to search the most similar antibody sequences in human germline. The closest matching candidates with compatible inter-chain interface residues and support loops with parental CDR canonical conformations were selected as the framework for CDR-grafting. The sequences of grafted CDRs on the selected human immunoglobulin framework were generated and further analyzed by Lonza's in silico Manufacturability Assessment platform to facilitate the full humanization and identify the sequence liabilities with possible post-translational modifications (PTM) on the designed humanized antibodies. To facilitate the full humanization, the substitutions of glutamate to glutamine at position 1 (E1Q) and lysine to threonine at position 74 (L74T) were created in some variants (Var_2, Var 4, Var_6, Var 8, Var_10, Var 12, Var_14 and Var 16) (shown in FIG. 1A). In some variants (from Var_5 to Var 16), the substitutions of asparagine to serine at position 30 (N30S) on the light chain and/or asparagine to serine at position 55 (N55S) on the heavy chain were designed to remove the PTM (shown in FIG. 1A). Lastly, the designed humanized antibody sequences were loaded into the Epibase® v. 4.0 immunoprofiling algorithm to evaluate the possible epitope binding in 85 HLA class II allotypes. The substitutions of lysine to arginine at position 38 (K38R), serine to lysine at position 76 (S76K) and valine to threonine at position 93 (V93T) on the heavy chain were made in different variants (Var_3, Var 4, Var_7, Var 8, Var_11, Var 12, Var_15 and Var 16) to reduce the possible immunogenicity in humans (shown in FIG. 1A).

### 2. Overexpression of 16 Humanized Variants and Chimeric Antibody

The proposed DNA fragments of different humanized variants based on in silico analyses were synthesized. The construct of heavy chain variable domain was generated by the insertion of synthesized DNA fragments into the vector pXC-IgG4proDK using restriction sites HindIII and ApaI. The construct of light chain variable domain is generated by the insertion of synthesized DNA fragments into the vector pXC-Kappa using restriction sites HindIII and BsiWI. Plasmids are transformed into the bacterial and isolation of plasmids with QIAGEN Gigaprep system (Qiagen, 12291) is performed to extract sufficient plasmid DNA for transient transfection in CHOK1SV GS-KO cells. CHOK1SV GS-KO cells were cultured in CD-CHO media (Life Technologies, 10743-029) supplemented with 6 mM L-glutamine (Life Technologies, 25030-123). For transfection, a total 400 μg of DNA was added to the flask followed by the addition of PEI Max at 1 mg/ml and sodium acetate to the final concentration of 10 mM. Cells were cultured at 32 degree, 5% CO₂, 85% humidity for 6 days. Supernatant was harvested by centrifugation at 2000 rpm for 10 minutes and filtered in 0.22 μm filter.

### 3. Purification of 16 Humanized Variants and Chimeric Antibody

Antibodies were purified from the supernatant using a pre-packed 5 ml HiTrap MabSelectSuRE column (Cytiva, 11003494) pre-equilibrated in binding buffer (50 mM sodium phosphate and 125 mM sodium chloride, pH 7.0) then washed with wash buffer (50 mM sodium phosphate and 1M mM sodium chloride, pH 7.0) and eluted with elution buffer (10 mM sodium formate, pH 3.5). Purified antibodies were collected in fractions of 10 ml into 5 ml PBS and 100 μl neutralization buffer (1 M TrisHCI, pH 9.0) and buffer exchange was performed overnight using 10 kDa Dialysis Cassettes (#87732, Thermofisher). Purified humanized variants were prepared for analysis by adding NuPAGE 4× sample buffer (Life Technologies, NP0007) with NuPAGE 10× sample reducing agent buffer (Life Technologies, NP0009), and incubated at 70 degree, 10 min. Samples were electrophoresed on 4-20% Mini-PROTEAN TGX stain-Free™ Precasts Gels (BioRad, 4568093) and imaged on a ChemiDoc XRS+ system (BioRad). (FIG. 1B)

### Example 2: Binding Activities of Humanized Variants Against huGREM1 and huGREM2

### Enzyme-Linked Immunosorbent Assay (ELISA)

An ELISA assay was used to analyze the anti-panGREM humanized variants for binding activity. High-binding 96-well polystyrene plates (Corning) were coated with either human Gremlin-1 or human Gremlin-2 conjugated to BSA (#77667, Thermofisher) in 0.1 M carbonate-bicarbonate buffer, pH 9.5. Plates were incubated overnight at 4° C. The plates were washed 4 times with PBS containing 0.05% Tween-20 (PBS-T) (Sigma-Aldrich) and blocked with 5% non-fat dry milk diluted in PBS (PBS-M) for 1 h at 37° C. After washing, anti-panGREM humanized variants were diluted in PBS-M and added to the wells. Plates were incubated for 1 h at 37° C., followed by four washes with PBS-T and then incubated for 1 h of at 37° C. with horseradish-peroxidase-conjugated goat-anti-human IgG4 antibodies (1:1000 in PBS-M, #99823 abcam). After four washes with PBS-T, ortho-phenylenediamine (0.5 mg/ml; Sigma) in 0.1 M citrate buffer, pH 5.6, containing 0.08% H₂O₂ was used to develop the reaction and the reaction was stopped after 10 minutes by adding 2.5 N Sulfuric acid. Optical density was measured at 492 nm using an automated ELISA plate reader (Tecan).

### Example 3: Neutralization Activity of Anti-panGREM Humanized Variants

Gremlin-1 and Gremlin-2 are potent inhibitors of BMP4 activity, e.g. when used in an in vitro stimulation assay that measures BMP4-mediated signaling. A neutralization assay was conducted to determine the neutralization activity of three anti-panGREM humanized variants Var 5, Var_7 and Var_13. Binding of BMP4 to type I and II receptors expressed on the chondrogenic cell line ATDC5 induces ALP production can be measured in a colorimetric assay at 405 nm, while addition of Gremlin-1 or Gremlin-2 inhibit binding of BMP4 to its receptor and therefore the production of ALP. The neutralizing capacity of anti-panGREM humanized variants prevent panGREM-mediated inhibition of BMP4 binding to its receptor, thus resulting in the production of ALP.

In Vitro panGremlin Neutralization Assay

ATDC5 cells were grown in DMEM:Ham's F12 (1:1) supplemented with 2 mM glutamine, 5% FCS. Cells were seeded in 96-well flat bottom plates at a concentration of 1.5×104 cells in 100 μl per well and incubated overnight at 37° C. On the following day, 50 μl of medium were gently removed from each well. In a separate 96-well round bottom plate, each antibody was serially diluted 1:2 and tested in triplicates. HuGREM-1 or huGREM-2 at a concentration of 0.9 μg/ml was added to the plate and mixed. This mixture was transferred to the plate containing the ATDC5 cells and incubated for 20 min at 37° C. After the incubation, 50 μl/well of huBMP4 at a concentration of 0.5 μg/ml was added. The plates were incubated for 24 h at 37° C. On the third day, the medium was gently flicked, and cells were washed twice with 200 μl of PBS. 50 μl of deionized water were added to each well and incubated for 5 min at room temperature. To determine the ALP activity, 100 μl p-nitrophenyle substrate were added to the plate and immediately measured in an ELISA reader (Tecan, OD 405 nm, 5 measurements every 2 minutes, 10 seconds shaking). For the analysis, the measurement in which the absorbance of the positive control (BMP4) was between 0.8 and 1 was chosen to calculate the neutralization activity of the anti-panGREM antibodies.

Addition of anti-panGREM humanized variants Var_5, Var_7 and Var_13 almost completely restored BMP4-mediated signaling indicating a highly efficient binding to Gremlin-1 and Gremlin-2 (FIG. 3A-B). Moreover, we found that the anti-panGREM humanized variants Var_5 and Var_7 showed an increased neutralizing activity compared to chimeric control and their parental mouse panGREM antibody 14-D10-2 demonstrating that the substitution of asparagine to serine at position 55 (N55S) on the CDR-2 of the heavy chain results in improved neutralization capacity.

### Example 4: Anti-panGREM Humanized Antibody Reduces Pericardial and Myocardial Inflammation, Systemic Inflammation Markers and Restores Cardiac Function

### Mouse Models of Myocarditis and Pericarditis

To investigate efficacy of the humanized pan-Gremlin antibody in preventing pericarditis and the progression to inflammatory cardiomyopathy, we used a rapid progression model of myocarditis. Importantly, concomitant to the cardiac inflammation, mice develop pericarditis that is characterized by the accumulation of immune cells in the pericardium and with the activation of pericardial fibroblast that form the niche for the autoimmune T cell proliferation.

Splenocytes from TCRM mice, which express a MYH6₆₁₄₋₆₂₉-specific T cell receptor in >95% of their cells (comprising a Vα2 and Vβ8 chain) (Nindl, V, et al., 2012 European journal of immunology 42.9: 2311-2321.) are adoptively transferred to Rag1tm1Mom (Rag1−/−) mice. All the mice present severe pericarditis and myocarditis 4 weeks after the adoptive transfer of TCRM cells. Importantly, this model compatible with the administration humanized antibodies as Rag1−/− mice cannot react against the humanized therapeutically agent due to the lack of endogenous adaptive immune cells.

Briefly, spleens were collected from TCRM mice and disrupted on a 70 μm cell strainer. Red blood cells were lysed by osmotic shock and 10⁶ splenocytes were injected intravenously in the lateral tail vein of 4-week-old Rag1−/− mice. Seven days after adoptive transfer, mice were bled to confirm CD4+ T cell expansion. Disease activity scores and analysis of T cell activation in the heart and pericardium were performed at day 28 post adoptive transfer. Mice were treated twice per week i.p. with 200 μg of the different 14-D10-2 humanized variants Var 3, Var 5, Var 7, Var_11 and/or Var_13 or human IgG4 as isotype control antibody. Treatment was initiated three days before the adoptive transfer of T cells.

### Cell Isolation and Flow Cytometry

Euthanized animals were perfused with 20 ml of PBS and small heart tissue pieces were placed into a six-well dish filled with RPMI 1640 medium containing 2% FCS, 20 mM HEPES (Lonza), 1 mg/ml collagenase D (Sigma), and 25 μg/ml DNase I (Applichem) and incubated at 37° C. under continuous stirring. The remaining tissue pieces were mechanically disrupted, and mononuclear cells were purified by centrifugation (25 min at 800×g, 4° C.) on a 30%-70% Percoll gradient (GE Healthcare). Single-cell suspensions were first stained with the fixable viability dye Zombie Aqua (Biolegend) and incubated for 30 min on ice, after washing, cells were incubated for 20 min at 4° C. in PBS containing 2% FCS and 10 mM EDTA with fluorochrome-labeled antibodies. Cells were acquired with a BD LSRFortessa (BD Biosciences) and analyzed using FlowJo software (Treestar Inc) following stablished guidelines.

### Ex Vivo Restimulation and Cytokine Production of Murine T Cells

For assessment of ex vivo production of IFN-γ and IL-17A, 10⁶ lymphocytes were incubated for 3.5 h at 37° C. in 96-well round-bottom plates in 200 μl of RPMI 5% FCS supplemented with 10 μg/ml brefeldin A (Sigma). Cells were stimulated with 0.25 μg of the MYH6₆₁₄₋₆₂₉ (RSLKLMATLFSTYASADR (SEQ ID NO: 37)) peptide or were left untreated. After surface molecule labelling (CD45, CD3, CD4 and Vβ8), cells were stained with the fixable viability dye Zombie Aqua (Biolegend). Cells were fixed with cytofix-cytoperm (BD Biosciences) for 20 min. Fixed cells were incubated at 4° C. for 40 min with permeabilization buffer (2% FCS, 0.5% saponin in PBS) containing antibodies to IFN-γ and IL-17A. Samples were measured using a BD LSR Fortessa (BD Biosciences). Data were analyzed using FlowJo software (Tree Star, Inc.).

### C-Reactive Protein Measurement by ELISA

For assessment of CRP, serum samples were obtained from Rag−/− mice adoptively transferred with 1×10⁶ TCRM splenocytes and treated with isotype antibody or the 14D10-2 humanized antibody variants twice a week for 28 days. Blood samples were obtained in BDMicrotainer tubes and centrifuged at 8500 RPM for 5 min. Serum samples were stored at −20° C. until analysis. CRP concentration was measured using the Quantikine Mouse CRP ELISA (MCRP00, R&D systems), following the manufacturer's instructions.

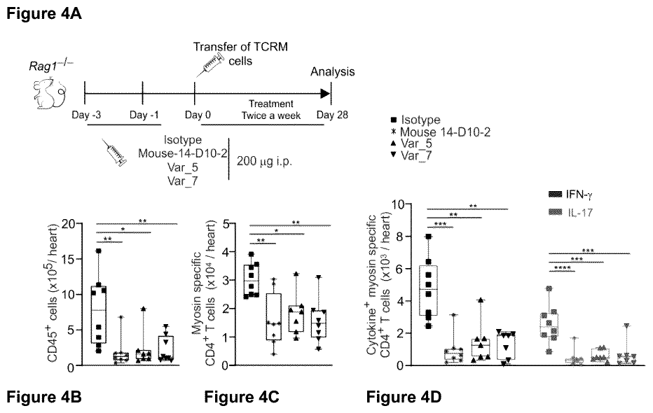

Rag1^{-/-} mice were treated with 200 μg of the humanized 14-D10-2 variants (Var_5, Var_7,) before the cell transfer of cardiac myosin-specific autoreactive CD4⁺ T cells; an irrelevant human IgG4 antibody (Isotype) and murine 14-D10-2 antibodies were used as controls (FIG. 4A). Overall inflammation was determined by quantifying CD45⁺ cell infiltration in the pericardial and cardiac tissue. Moreover, the effect of prophylactic treatment with 14-D10-2 and its humanized variants was determined based on the accumulation of cardiac myosin-specific autoreactive CD4⁺ T cells in the heart and the ability of these pathogenic CD4⁺ T cells to produce IFN-γ and IL-17A. Rag1^{-/-} mice treated prophylactically with 200 μg of Var_5 and Var_7 showed significantly reduced overall inflammation as determined by infiltration of CD45+ cells in cardiac tissues (FIG. 4B; Var_5 p=0.01, Var_7 p=0.007). Moreover, the prophylactic treatment with Var_5 and Var_7 significantly reduced the accumulation of cardiac myosin-specific autoreactive CD4⁺ T cells in the heart (FIG. 4C; Var_5 p=0.04, Var_7 p=0.007) and the ability of these pathogenic CD4⁺ T cells to produce IFN-γ (FIG. 4D; Var_5 p=0.002, Var_7 p=0.001) and IL-17A (Var_5 p=0.0009, Var_7 p=0.001).

### Example 5 Prevention of Asparagine De-Amidation of Humanized panGREM Antibody Variants Increases in Vivo Half-Life

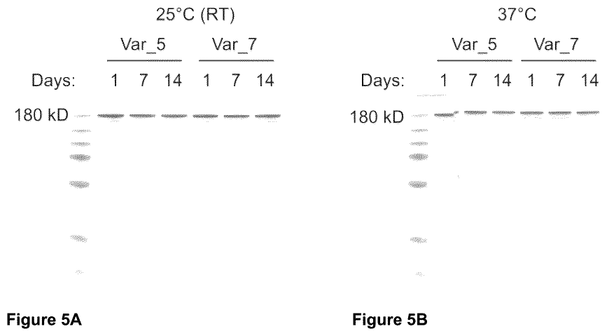

Some humanized anti-GREM1/2 were modified to prevent asparagine de-amidation through the replacement of the key amino acid residue asparagine with serine (N55S Kabat numbering) in the heavy chain (FIG. 1A). This modification method may affect the antibody stability under conditions of low or high pH, increased temperature, or fluctuated temperature (Pace, Amanda L et al. Journal of pharmaceutical sciences vol. 102, 6 (2013): 1712-1723.; Gupta, Surbhi et al. Journal of pharmaceutical sciences vol. 111, 4 (2022): 903-918.). We assessed antibody stability of different humanized variants (Var_5 and Var_7) with exchange of residue asparagine to serine (N55S) through incubation at 25° C. or 37° C. for two weeks. The antibody degradation or disassembly of heavy and light chains was examined by non-reducing SDS PAGE. Our results did not show any differences following storage at different temperatures among different 14-D10-2 variants when analyzed by SDS PAGE (FIGS. 5A and B).

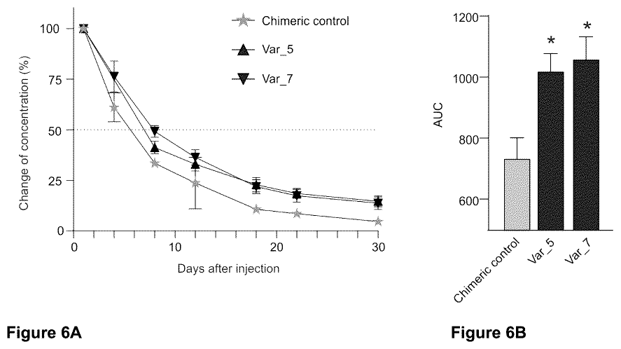

In order to test if the replacement of the asparagine residue with serine (N55S) to prevent asparagine de-amidation affects the pharmacokinetics of the antibodies, we treated Rag1^{-/-} mice intraperitoneally with 200 μg of chimeric 14-D10-2, Var_5 or Var_7. The mice were bled on days 1, 2, 4, 6, 8, 10, 12, 14, 16, 18, 22 and 24 after antibody injection. The presence of the humanized anti-GREM1/2 variants Var_5 and Var_7 in circulation was determined by quantifying human-IgG4 concentration in the serum at the indicated time points following antibody administration (FIG. 6A). The half-life of each antibody was calculated as the time point when a 50% reduction in serum concentration compared to the initial circulating IgG4 concentration could be determined. Changes in the pharmacokinetics of each antibody were calculated as the area under the curve (AUC) and statistically evaluated by One-way ANOVA with Dunnett's posttest. Var_5 (half-life 7.2 days) and V_7 (8.2 days) demonstrated a significantly increased half-life when compared with the chimeric control (5.7 days) (FIG. 6B; Var_5 p=0.04, Var_7 p=0.03).

1. An antibody, or an antigen-binding fragment thereof, pan-specifically binding to Gremlin-1 and Gremlin-2.

2. The antibody, or antigen-binding fragment thereof, according to claim 1, wherein the antibody or antigen-binding fragment thereof, binds to Gremlin-1 within the amino acid sequence SEQ ID NO: 1 and/or to Gremlin-2 within the amino acid sequence SEQ ID NO: 2.

3. The antibody, or antigen-binding fragment thereof, according to claim 2, wherein the antibody or antigen-binding fragment thereof, binds to Gremlin-1 within the amino acid sequence SEQ ID NO: 1 and to Gremlin-2 within the amino acid sequence SEQ ID NO: 2.

4. The antibody, or antigen-binding fragment thereof, according to any one of the claims 1 to 3, comprising at least one CDR as defined by the SEQ ID NO: 4.

5. The antibody, or antigen-binding fragment thereof, according to any one of the claims 1 to 3, comprising at least one CDR as defined by the SEQ ID NO: 10.

6. The antibody, or antigen-binding fragment thereof, according to any one of claims 1 to 5, comprising
(a) a variable heavy (VH) chain comprising CDR1 as defined in SEQ ID NO: 3, CDR2 as defined in SEQ ID NO: 4 and CDR3 as defined in SEQ ID NO: 5 and a variable light (VL) chain comprising CDR1 as defined in SEQ ID NO: 6, CDR2 is GAT and CDR3 as defined in SEQ ID NO: 8;
(b) a variable heavy (VH) chain comprising CDR1 as defined in SEQ ID NO: 3, CDR2 as defined in SEQ ID NO: 9 and CDR3 as defined in SEQ ID NO: 5 and a variable light (VL) chain comprising CDR1 as defined in SEQ ID NO: 10, CDR2 is GAT and CDR3 as defined in SEQ ID NO: 8; or
(c) a variable heavy (VH) chain comprising CDR1 as defined in SEQ ID NO: 3, CDR2 as defined in SEQ ID NO: 4 and CDR3 as defined in SEQ ID NO: 5 and a variable light (VL) chain comprising CDR1 as defined in SEQ ID NO: 10, CDR2 is GAT and CDR3 as defined in SEQ ID NO: 8.

7. The antibody, or antigen-binding fragment thereof, according to any one of claims 1 to 6, wherein the antibody or the antigen-binding fragment thereof comprises
(a) a variable heavy (VH) chain sequence comprising the amino acid sequence of SEQ ID NO: 12 or a sequence having at least 90%, preferably at least 95% sequence identity to SEQ ID NO: 12; and
a variable light (VL) chain sequence comprising the amino acid sequence of SEQ ID NO: 17 or a sequence having at least 90%, preferably at least 95% sequence identity to SEQ ID NO: 17;
(b) a variable heavy (VH) chain sequence comprising the amino acid sequence of SEQ ID NO: 13 or a sequence having at least 90%, preferably at least 95% sequence identity to SEQ ID NO: 13; and
a variable light (VL) chain sequence comprising the amino acid sequence of SEQ ID NO: 18 or a sequence having at least 90%, preferably at least 95% sequence identity to SEQ ID NO: 18;
(c) a variable heavy (VH) chain sequence comprising the amino acid sequence of SEQ ID NO: 14 or a sequence having at least 90%, preferably at least 95% sequence identity to SEQ ID NO: 14; and
a variable light (VL) chain sequence comprising the amino acid sequence of SEQ ID NO: 19 or a sequence having at least 90%, preferably at least 95% sequence identity to SEQ ID NO: 19;
(d) a variable heavy (VH) chain sequence comprising the amino acid sequence of SEQ ID NO: 15 or a sequence having at least 90%, preferably at least 95% sequence identity to SEQ ID NO: 15; and
a variable light (VL) chain sequence comprising the amino acid sequence of SEQ ID NO: 20 or a sequence having at least 90%, preferably at least 95% sequence identity to SEQ ID NO: 20;
(e) a variable heavy (VH) chain sequence comprising the amino acid sequence of SEQ ID NO: 11 or a sequence having at least 90%, preferably at least 95% sequence identity to SEQ ID NO: 11; and
a variable light (VL) chain sequence comprising the amino acid sequence of SEQ ID NO: 16 or a sequence having at least 90%, preferably at least 95% sequence identity to SEQ ID NO: 16.

8. The antibody, or antigen-binding fragment thereof, according to any one of claims 1 to 7, wherein the antibody, or antigen-binding fragment thereof, is a humanized antibody or a humanized antigen-binding fragment thereof.

9. The antibody, or antigen-binding fragment thereof, according to claim 8, wherein the antibody, or antigen-binding fragment thereof, is a deimmunized antibody or a deimmunized antigen-binding fragment thereof.

10. The antibody, or antigen-binding fragment thereof, according to claim 8 comprising at least one modified amino acid and/or defined amino acid selected from the group of HC 54S, HC 38R, HC 75K, HC 89T, LC 30S, defined by Chothia scheme; and/or the group of HC 55S, HC 38R, HC 76K, HC 93T, and LC 30S, defined by Kabat numbering scheme.

11. A polynucleotide encoding an antibody, or an antigen-binding fragment thereof, according to any one of claims 1 to 10.

12. A host cell comprising the polynucleotide of claim 11.

13. A method for producing an antibody comprising culturing the host cell of claim 12.

14. A pharmaceutical composition comprising the antibody, or antigen-binding fragment thereof, according to any one of claims 1 to 10, the polynucleotide of claim 11 or the host cell of claim 12, and a pharmaceutically acceptable carrier.

15. The pharmaceutical composition according to claim 14, comprising at least one further therapeutic agent.

16. The pharmaceutical composition according to claim 15, wherein the further therapeutic agent is selected from the group consisting of an anti-inflammatory agent, an immunomodulator, an antibiotic, an angiotensin-converting-enzyme inhibitor, a 3-blocker and a diuretic.

17. The pharmaceutical composition according to claim 15 or 16, wherein the further therapeutic agent is
(i) an anti-inflammatory agent selected from the group consisting of infliximab, adalimumab, certolizumab pegol, golimumab, etanercept, curcumin, IL-1 RA, rilonacept, canakinumab, allopurinol, colchicine, prednisone, pentoxifylline, rosuvastatin and oxypurinol;
(ii) an immunomodulator selected from the group consisting of antigenic peptide, immunoglobulin, methotrexate and stem cell-based therapy; or
(iii) an antibiotic selected from the group consisting of anti-bacterial phages, rifaximin, vancomycin, and trimethoprim-sulfamethoxazole.

18. The antibody, or antigen-binding fragment thereof, of any one of claims 1 to 10, the polynucleotide of claim 11 or the host cell of claim 12, or the pharmaceutical composition of any one claims 14 to 18 for use in treating and/or preventing heart failure and/or an inflammatory disease.

19. The antibody, or antigen-binding fragment thereof, for use of claim 18, the polynucleotide for use of claim 18, or the host cell for use of claim 18, or the pharmaceutical composition for use of claim 18, wherein the inflammatory disease is an inflammatory disease of the heart, in particular inflammatory dilated cardiomyopathy, inflammatory cardiomyopathy, myocarditis, pericarditis, perimyocarditis or myopericarditis.

20. The antibody, or antigen-binding fragment thereof, for use of claim 18, the polynucleotide for use of claim 18, or the host cell for use of claim 18, or the pharmaceutical composition for use of claim 18, wherein the inflammatory disease is reperfusion injury, allergy, asthma, coeliac disease, glomerulonephritis, hepatitis, inflammatory bowel disease or transplant rejection.

21. The antibody, or antigen-binding fragment thereof, of any one of claims 1 to 10, the polynucleotide of claim 11 or the host cell of claim 12, or the pharmaceutical composition of any one of claims 14 to 18 for use in the treatment and/or prevention of a disease or disorder associated with SARS-CoV-2 infection and/or SARS-CoV-2 vaccination.

22. The antibody, or antigen-binding fragment thereof, for use of claim 21, the polynucleotide for use of claim 21, or the host cell for use of claim 21, or the pharmaceutical composition for use of claim 21, wherein the disease or disorder associated with SARS-CoV-2 infection and/or SARS-CoV-2 vaccination is heart failure and/or an inflammatory disease of the heart.
